(19) United States
(12) Patent Application Publication
(10) Pub. No.: US 2011/0250613 A1
(43) Pub. Date: Oct. 13, 2011

(54) Arrayed Multiple Ubiquitin Binding Domains as Linkage-specific Polyubiquitin Affinity Reagents

(75) Inventors: Joshua J. Sims, Baltimore, MD (US); Robert E. Cohen, Baltimore, MD (US)

(73) Assignee: THE JOHNS HOPKINS UNIVERSITY, Baltimore, MD (US)

(21) Appl. No.: 12/815,740

(22) Filed: Jun. 15, 2010

Related U.S. Application Data
(60) Provisional application No. 61/187,104, filed on Jun. 15, 2009.

(51) Int. Cl. G01N 33/566; C12N 9/00; C12N 1/21; C07K 14/00

(52) U.S. Cl. 435/7.1; 530/350; 435/183; 435/252.33; 436/501

(57) ABSTRACT

Linkage-specific polyubiquitin recognition is thought to make possible the diverse set of functional outcomes associated with ubiquitination. Thus far, mechanistic insight into this selectivity has been largely limited to single domains that preferentially bind to lysine 48-linked polyubiquitin (K48-polyUb) in isolation. A mechanism is proposed herein, linkage-specific avidity, in which multiple ubiquitin-binding domains are arranged in space so that simultaneous, high-affinity interactions are optimum with one polyUb linkage but unfavorable or impossible with other polyUb topologies and monoUb. The model used herein is human Rap80, which contains tandem ubiquitin interacting motifs (UIMs) that bind to K63-polyUb at DNA doublestrand breaks. The sequence between the Rap80 UIMs positions the domains for efficient avid binding across a single K63 linkage, thus defining selectivity. K48-specific avidity is also demonstrated in a different protein, ataxin-3. Using tandem UIMs, the general principles governing polyUb linkage selectivity and affinity in multivalent ubiquitin receptors are established.

### CROSS-REFERENCE TO RELATED APPLICATION

This application claims the benefit under 35 U.S.C. §119(e) of U.S. Provisional Application Ser. No. 61/187,104, filed Jun. 15, 2009, which is entirely incorporated herein by reference.

### STATEMENT OF GOVERNMENTAL INTEREST

This invention was made with U.S. government support under grant no. NIH U54 RR020839 and grant no. NIH GM065334. The U.S. government has certain rights in the invention.

### FIELD OF THE INVENTION

The invention relates to ubiquitin. More specifically, the present invention relates to the design of affinity reagents that can distinguish topologically distinct types of polyubiquitin.

### BACKGROUND OF THE INVENTION

The covalent attachment of the small protein ubiquitin (Ub) to other proteins is an essential step in an enormous variety of cellular processes (Pickart and Eddins, 2004). Substrates can be modified with a single Ub unit or polymeric Ub chains assembled by the linkage of one Ub C terminus to any of seven lysines on another Ub molecule (Peng et al., 2003). Whereas ubiquitination with chains linked though lysine 48 (K48-polyUb) leads to degradation of the substrate protein at the 26S proteasome (Pickart and Cohen, 2004), lysine 63-linked polyUb and monoUb function as distinct but nonproteolytic signaling elements (Sun and Chen, 2004) in pathways such as endocytosis, DNA repair, DNA damage tolerance, NF-κB signaling, and translation. The prevailing model holds that this functional diversity is possible because downstream receptors can distinguish the Ub forms by selective binding (Pickart and Fushman, 2004).

### SUMMARY OF THE INVENTION

The present invention discloses a novel mechanism, linkage-specific avidity, in which multiple ubiquitin binding domains (UBDs) are arranged in space so that simultaneous, high-affinity interaction are optimum with only poly Ub linkage, but unfavorable or impossible with other polyUb topologies and monoUb. A UBD refers to a protein domain that independently recognizes and interacts with Ubiquitin. UBDs are typically about 20 to about 40 amino acid long structural motifs and are found in all eukaryotes.

In certain embodiments, the UBD is a UIM or Ubiquitin Interacting Motif. As described herein, tandem UIMs were used to establish the general principles governing polyUb linkage selectivity and affinity in multivalent ubiquitin receptors. Linkage-specific avidity explains how selective binding is achieved by many physiological polyubiquitin receptors. In a particular embodiment, tandem ubiquitin interacting motifs (UIMs) spaced with a seven amino acid linker are efficient, avid binding proteins selective for K63 linkages. In another embodiment, reducing the linker length to 2 amino acids confers K48-specific avid binding.

Implementation of the linkage-specific avidity model described herein makes possible the construction of particular proteins. In one aspect, the methods and compositions of the present invention are useful for the detection of specific forms of polyubiquitin in vitro and in vivo. In another aspect, the methods and compositions of the present invention are useful in constructing specific inhibitors of ubiquitin-dependent pathways. In yet another aspect, the present invention is useful in constructing new protein useful in modifying ubiquitin pathway enzymes to have altered substrate specificities.

Thus, in one aspect, the present invention provides polypeptides having linkage-specific avidity for polyubiquitinated proteins. In one embodiment, the proteins of the present invention have linkage-specific avidity for K63 polyubiquitinated proteins. In another embodiment, the proteins of the present invention have linkage-specific avidity for K48 polyubiquitinated proteins. Linkage-specific avidity requires no specific contact at or near the isopeptide bond of the polyubiquitinated proteins.

In particular embodiments, a polypeptide having linkage-specific avidity for K-63 polyubiquitinated proteins may comprise at least two ubiquitin binding domains (UBDs) linked to each other by an α-helical amino acid sequence. The amino acid linker of the polypeptide may comprise about 2 to about 10 amino acids. In a specific embodiment, the amino acid linker comprises 8 amino acids. In another embodiment, the amino acid linker comprises 7 amino acids.

In certain embodiments, the UBDs are the same or different. Indeed, the present invention is also applicable to other ubiquitin binding domains to achieve linkage-selective avid binding. More specifically, the UBDs may be selected from the group consisting of UIM (Ubiquitin Interacting Motif), UBA (Ubiquitin Associated domain), UBM (Ubiquitin Binding Motif), MIU (Motif Interacting with Ubiquitin), DUIM (Double-sided Ubiquitin Interacting Motif), CUE (Coupling of Ubiquitin Conjugation to ER degradation), UBZ (Ubiquitin-Binding Zinc Finger), NZF (Np14 Zinc Finger), A20 ZnF (Zinc Finger), UBP Znf (Ubiquitin-specific Processing Protease Zinc Finger), UEV (Ubiquitin-conjugating Enzyme E2 variant), PFU (PLAA Family Ubiquitin binding), GLUE (GRAM-Like Ubiquitin binding in EAP45), GAT (Golgi-localized, Gamma-ear-containing, Arf-binding), Jab/MPN (Jun kinase Activation domain Binding/Mpr1p and Pad1p N-termini), and a Ubc (Ubiquitin-Conjugating enzyme). In particular embodiments, the UBDs are UIM. Moreover, the UBDs may be derived from the Rap80 protein or the ataxin-3 protein.

In a specific embodiment, the present invention provides a polypeptide having linkage-specific avidity for K-63 polyubiquitinated proteins comprising at least two ubiquitin interacting motifs (UIMs) linked to each other by an amino acid sequence that adopts a helical conformation. Alternatively, a polypeptide having linkage-specific avidity for K-63 polyubiquitinated proteins may comprise at least two ubiquitin interacting motifs (UIMs) linked to each other by an amino acid sequence that reduces flexibility between the UIMs.

The amino acid linker may comprise about 2 to about 10 amino acids. In a specific embodiment, the amino acid linker comprises 8 amino acids. In another embodiment, the amino acid linker comprises 7 amino acids. In a specific embodiment, a polypeptide having linkage-specific avidity for K-63 polyubiquitinated proteins may comprise tandem UIMs linked by a seven amino acid sequence. In certain embodiments, the UIMs are the same or different. The UIMs may be derived from the Rap80 protein or the ataxin-3 protein.

A polypeptide of the present invention may further a detection tag. In other embodiments, a host cell may comprise a polynucleotide sequence encoding a polypeptide of described herein. Furthermore, the present invention provides methods for identifying, isolating, and/or purifying polyubiquitinated proteins. In one embodiment, a method for isolating K63 polyubiquitinated proteins may comprise the steps of contacting a polypeptide of the present invention with at least one candidate K63 polyubiquitinated protein under conditions allowing the interaction between the UBDs or UIMs of the polypeptide with the ubiquitin molecules of the candidate K63 polyubiquitinated protein, and detecting the interaction. The polypeptide may comprise a detectable tag. Alternatively, the candidate protein may comprise a detectable tag.

In a specific embodiment, the present invention provides a polypeptide having linkage-specific avidity for K-48 polyubiquitinated proteins comprising at least two UIMs linked to each other by two amino acids. In an alternative embodiment, the polypeptide having linkage-specific avidity for K-48 polyubiquitinated proteins may comprise tandem UIMs linked by a two amino acid sequence. The UIMs may be the same or different. The UIMs may be derived from the Rap80 protein or the ataxin-3 protein.

### BRIEF DESCRIPTION OF THE FIGURES

As shown in FIG. 1, binding studies with Rap80 UIM peptides reveal that differences in avid binding underlie K63-linked polyUb-binding selectivity. (A) The Rap80 protein is shown schematically with the UIMs indicated in blocks (top). The sequences of the tandem UIM (tUIM) and single UIM peptides are shown each with the linker sequence underlined and the cysteine residue used for fluorescent labeling shaded gray (bottom). (B) Coomassie staining (left) and fluorescence image (right) of purified Rap80 tUIM peptide after SDS-PAGE. (C-E) Fluorescence anisotropy binding data for the Rap80 tUIM peptide (filled circles), UIM1 (gray squares), and UIM2 (gray triangles) titrated with monoUb (C), K63-Ub₂ (D), or K48-Ub₂ (E). Schematic binding models for the interaction of each ubiquitin species with the tUIM peptide are inset in each plot. (F and G) Isothermal titration calorimetry measurements for the Rap80 tUIM peptide with K63-Ub₂ (F) or K48-Ub₂ (G). Raw data traces are inset in the integrated, fit data plots.

As depicted in FIG. 2, modeling and structural measurements reveal the molecular basis of linkage-specific avidity for the Rap80 tUIM peptide. (A) The structures of live UIM domains (S5a UIM1 from 1YX5.pdb [red', S5a UIM2 from 1YX6.pdb [blue', Vps27 UIM1 from 1Q0W.pdb [green', and the double-sided UIM from Hrs, 2D3G.pdb [orange and yellow]) bound to monoUb (gray) were aligned using the Ub coordinates from each complex. The Ub coordinates from 1QOW.pdb are shown. The ubiquitin hydrophobic patch (Sloper-Mould et al., 2001) is shown in light gray spheres for all models. Two perspectives show the common UIM orientation on the surface of ubiquitin with respect to the C terminus and K63 (dark gray spheres). (B) The homology model of unbound Rap80 tUIMs shows an unstructured linker. (C) The model for Rap80 (green) bound to K63-linked diUb (gray) shows how a helical linker orients the UIM domains for optimum simultaneous interactions. The isopeptide bond is shown between the C terminus and K63 (dark gray spheres). (D) CD spectra for 153 mM Rap80 alone (triangles), 153 mM K63-Ub₂ alone (squares), and a mixture of 153 mM Rap80 with 153 mM K63-Ub₂ (circles). The sum of the unbound Rap80 and K63-Ub₂ spectra (crosses) is shown for comparison to the bound complex. (E) An alignment of all tested 7-residue tUIM linkers that support K63-specific binding shows no sequence conservation.

FIG. 3 demonstrates that binding and structural measurements reveal the factors governing polyUb linkage specificity and affinity for tUIMs. (A) Summary of fluorescence anisotropy binding data collected for Rap80 tUIMs with various all-alanine linkers interacting with K63-Ub₂. (B) The pattern of K63-Ub₂ affinities demonstrates that linker length can dramatically modulate linkage specificity by controlling the orientation of the UIM domains with respect to each other, thus affecting avidity. (C) CD spectra for various tUIM constructs establish the link between structure and polyUb binding for tUIMs. (D) An alignment of tUIMs from humans shows diverse linker lengths. Linkers that can easily adopt helical conformations are expected to follow the K63-polyUb binding pattern presented in (B).

FIG. 4 shows that the fluorescein label on Rap80 tUIM peptides does not influence ubiquitin binding. (A) In the presence of a saturating concentration of K63-Ub₂, the fluorescent Rap80 tUIM peptide (see FIG. 1) was titrated with an unlabeled version of the same peptide. The Ki for the binding inhibition (24 μM) was near the K_{d} measured for association by the fluorescent peptide (22 μM), indicating that the fluorescein does not influence ubiquitin binding. (B) A version of the Rap80 tUIM peptide was produced with no 6×His6-tag and with fluorescein covalently linked to a C-terminal lysine side chain by expressed protein ligation (Scheibner, Zhang, and Cole, 2003). This peptide was titrated with K63-Ub₂. The K_{d} (19 μM) was near the K_{d} measured for the cysteine-labeled, 6×His-tagged peptide (22 μM, FIG. 1), suggesting that neither the 6×His-tag nor the cysteine modification influence ubiquitin binding.

FIG. 5 demonstrates that complex divalent-divalent interactions are possible between Rap80 tUIM peptide and K63-Ub₂. (A) Whereas single-site fits (FIG. 1) only account for the tightest avid or “in-register” mode of binding, non-avid or “out-of-register” interactions are possible between two divalent molecules. This schematic of the complex interactions can be compared to FIG. 1D. (B) Binding data for the Rap80•K63-Ub₂ interaction were collected to high K63-Ub₂ (ligand) concentrations and fit to the binding equations for the model schematically depicted in (A) (Bobrovnik, 2007). Fluorescent Rap80 was present at 1 μM. Because avid and non-avid K_{d} values are not sufficiently separated, a saturation plateau is not observed. The contributions from each species are differentiated by color in the plot; the composite fit is black. The non-avid mode (blue) only contributes significantly to the observed anisotropy above 250 μM Ub₂; therefore, single-site fits (including FIG. 1) only include data up to this concentration. The avid K_{d} from the complex fit [in FIG. 4A, K_{d}^{avid}=(K₁*)(Kᵢₙₜᵣₐ)] was 22 μM, in exact agreement with the single-site fit (also 22 μM, FIG. 1). For the complex fit, the nonavid K_{d} (K₁* and K₂ from FIG. 5A) was 1000 μM, which agrees well with the measured interactions of single UIMs with monoubiquitin. (C) Fluorescent K63-Ub₂ (K63-Ub₂-fluor) was produced by mutating Ser20 of the proximal Ub to Cys, and labeling the resulting protein with fluorescein-5-maleimide. K63-Ub2-fluor (1 μM) was titrated with unlabeled Rap80 tUIM peptide. Unbound K63-Ub₂-fluor (the species present from 0.1 μM to 1 μM Rap80) has an intrinsic anisotropy that is higher than avidly-bound K63-Ub₂ (binding progresses between 1 μM and 250 μM Rap80), probably because the avid interaction restricts Ub-Ub domain movements and gives the molecule a smaller axis of rotation. At Rap80 tUIM concentrations >250 μM, non-avid interactions begin to displace the avid interactions, the restriction on the K63-Ub₂ conformation is released, and the anisotropy rapidly increases.

FIG. 6 shows that Rap80 tandem UIMs function independently of surrounding sequence to bind polyUb in the larger Rap80 protein context. (A) Although Rap80 is predicted to be a largely unstructured protein, sequences that surround the tandem UIM domains could influence polyUb binding. To test this possibility, the N-terminal 233 residues of Rap80 was expressed and purified and the protein was fluorescently labeled on the same UIM cysteine as with the isolated tUIM peptide. Coomassie staining (left) and fluorescence image (right) of purified, fluorescent Rap80 1-233 after SDS-PAGE are shown. Molecular weight standards (kDa) are to the left. (B) Fluorescence anisotropy binding data for Rap80 (1-233) (circles) and the minimal Rap80 tUIM construct (squares) interacting with K63-Ub₄ (black) or K48-Ub₄ (red). The longer Rap80 fragment exhibited essentially the same affinities and selectivities as the minimal tUIM peptide. K_{d} values are listed in (C). (C) The indicated Rap80 fragments were purified, fluorescently labeled, and used in fluorescence anisotropy binding assays to examine linkage preferences for tetraubiquitin chains. Smaller constructs revealed no obvious influence on polyUb binding by the sequences N- or C-terminal to the tUIMs. Thus, most of the elements of selectivity in full-length Rap80 are likely contained within the tUIM fragment. All binding assays were performed in fluorescence buffer with 0.1 μM fluorophore present.

As shown in FIG. 7, two ubiquitin units (grey) were modeled to interact with Rap80 tUIM configured as a single α-helix (green) using the consensus orientation from reported UIM•monoUb structures (see Examples and FIG. 2). The resulting conformations of the two ubiquitin units with respect to each other matched the known structure of the K63-Ub₂ dimer (Varadan et al., 2004), leading to the conclusion that the avidly-bound K63-Ub₂ can take this form. The dark grey spheres indicate residues K63 and G76 of the proximal and distal ubiquitins, respectively. The ubiquitin hydrophobic patch residues, which are critical for binding to UIMs, are shown as grey spheres. The only difference in the two sets of diubiquitin conformations is a minor rotation about the flexible isopeptide bond or the distal Ub C-terminus used to align the ubiquitin hydrophobic patches.

Although K48-Ub₂ predominantly adopts a closed conformation in solution that occludes both ubiquitin hydrophobic patches, a wide range of open conformations are possible that allow receptor binding (Varadan, Walker, et al, 2002). As shown in FIG. 8, the open-form coordinates from a bound K48-Ub₂ structure (Varadan et al., 2005) were chosen to model tUIM interactions. Consistent with the binding data, simultaneous UIM interactions could not be modeled to these K48-Ub₂ coordinates, even with an unstructured UIM linker. This figure depicts the interaction of Rap80 UIM1 with K48-Ub₂. The model also reveals a possible explanation for the observation that the single-UIM constructs bind K48-Ub₂ more tightly than K63-Ub₂. A UIM helix bound between the partially closed K48-linked ubiquitins could be sterically “caged” in the binding site by non-specific contacts that make rebinding after dissociation slightly more efficient.

FIG. 9 shows the results of estimating the helical content of Rap80 tUIM in complex with K63-Ub₂. Measurements of 3 samples were collected and are shown in FIG. 2D: (1) 153 μM Rap80 tUIM alone, (2) 153 μM K63-Ub₂ alone, and (3) a mixture of 153 μM Rap80 tUIM with 153 μM K63-Ub₂. The contribution of Rap80 tUIM to spectrum (3) was sought, so that the helical content of the bound tUIM could be estimated. To arrive at this, it was assumed that Ub₂ secondary structure does not change upon binding Rap80. Therefore, the difference between spectrum (3) and spectrum (2) will give the contribution from Rap80 tUIM to the total CD spectrum of the Rap80-K63-Ub₂ mixture. (A) The spectrum of unbound Rap80 [spectrum (1)] is shown here as triangles. The contribution of Rap80 to spectrum (3) was calculated as (3)−(2), and is shown here as squares. Because the mixture of Rap80 with K63-Ub₂ is not at saturating concentrations, bound and unbound Rap80 are both present in the solution. The observed helical fraction (Hₘᵢₓ) for this complex mixture can be described by:

Hₘᵢₓ=(F_{bound})(H_{bound})+(F_{unbound})(H_{unbound}),

Where F_{bound} and F_{unbound} are the fractions of the bound and unbound forms of Rap80 present in the mixture, respectively, and H_{bound} and H_{unbound} are the helical contents of the bound and unbound forms of Rap80, respectively. F_{bound} and F_{unbound} are calculated from the K_{d} of the interaction (22 μM) and the concentrations of the species (153 μM each). Hₘᵢₓ and H_{unbound} can be derived in a variety of ways from the two spectra shown in this figure, so that this equation may be solved for H_{bound}. Several methods were used to estimate the helical content of the spectra shown here. The K2D (Andrade, Chacon, et al, 1993) algorithm uses a neural-network trained on sets of spectra from proteins of known structure to estimate secondary structure from CD spectra. Simpler methods use only the molar ellipticity at 222 nm to determine α-helical content by comparing it to a theoretical value (Rohl, Chakrabartty, and Baldwin, 1996). (B) Shown here is a summary of the secondary structure predictions from two methods and the corresponding H_{bound} values calculated as described above. It is clear that Rap80 tUIM acquires significant helical structure upon binding K63-Ub₂, as the present model predicts.

FIG. 10 relates to the predictions and measurements of the helical content in various tandem UIM linkers. (A) Three secondary structure prediction algorithms were used (Di Francesco, Garnier, and Munson, 1996; Meiler, and Baker, 2003; Rost, Yachdav, and Liu, 2004) to analyze the sequences of Rap80 tUIMs linked by several different 7-residue sequences. The variations shown here all contain linking sequences that are predicted to be more helical than the wild-type Rap80 linker. As described herein, all of these peptides bind K63-Ub₂ more tightly than the wild-type Rap80 tUIM. (B) Shown here is the CD spectrum for wild-type Rap80 tUIM peptide. The depth of the trough at 222 nm in CD spectra is proportional to α-helix content (Greenfield, 2006). θ_{222 nm} values for closely related peptides can be compared directly to assess differences in helical content. (C) At 25° C., the θ_{222 nm} values for the set of peptides described in (A) were not significantly different (data not shown); however, when wild-type Rap80 tUIM was compared to the S101A variant at a lower temperature, the peptides were more helical and differences in helical propensity were clear, confirming the small predicted helicity difference (at 5° C., the θ_{222 nm} for the wild-type peptide was 19245 deg cm² dmol⁻¹; for S101A, θ_{222 nm}=20383 deg cm² dmol⁻¹; p=0.002). CD measurements were performed as described herein. These measurements confirm the role of tandem UIM linker structure in achieving linkage-specific avidity, and support the present model of Rap80 bound to K63-Ub₂.

As shown in FIG. 11, alignments of tUIM Proteins show the conservation in linker length. (A) Ataxin-3 homologs that contain UIM domains have a conserved 2-residue linker. Defining UIM residues are in red; linker residues are in grey. (B) A less diverse set of Rap80 homologs was available for alignment, but all mammalian Rap80 UIMs are separated by 7 residues. The tetraodon sequence shows an 8-residue linker, a length that the present studies indicate also supports high affinity K63polyUb binding.

FIG. 12 shows that GST-Ede1 UBA is selective for K63-linked polyUb. SPR analysis of GST-Ede1 binding to K63- and K48-linked (A) Ub₂ and (B) Ub₄ reveals preference for K63-linked polyUb (upper panels). The residuals (lower panels) indicate a systematic deviation from the 1:1 binding model used to fit the K63-linked polyUb data. These data are the averages of three or four independent experiments, except K48-Ub₄, which is the average of two trials. (C) GST-Ede1 was used to pull-down radiolabeled Ub4 chains (upper panel). The lower panel shows even loading of GST-Ede1 or control GST onto the glutathione beads. The averages of three such experiments are quantified in (D). The error bars indicate standard deviations for the three trials.

FIG. 13 demonstrates that CSP mapping of the interactions of Ede1 UBA with monoUb, K63-polyUb and K48-polyUb reveals no linkage-specific mode of interaction. Amide CSPs at titration endpoints are shown as a function of residue number for the titrations of ¹⁵N-Ede1 UBA with (A) monoUb, (B) K63-Ub₂, and (C) K48-Ub₂ (upper panels). Residues that were significantly perturbed upon binding (Δδ>0.15 ppm, or signal attenuation >60%) are mapped to the Ede1 UBA structure (2g3q.pdb) in red spheres below each plot. E1364 and K1365 are shown in cyan in (B). Likewise, Ub₂ molecules were segmentally labeled with ¹⁵N and titrated with unlabeled Ede1 UBA. Ub residues that were significantly perturbed (Δδ>0.1 ppm, or signal attenuation >60%) are mapped to the surface of Ub¹² (2g3q.pdb) in red for the (D) distal and (E) proximal K48-linked Ubs, and for the (F) distal and (G) proximal K63-linked Ubs. The CSP values are shown in FIG. 20, along with ¹⁵N monoUb CSPs, which are highly similar to both polyUb measurements.

FIG. 14 demonstrates that free Ede1-UBA is not linkage selective. Binding to K63- and K48-linked (A) Ub₂ and (B) Ub₄ were detected by monitoring the fluorescence anisotropy of Ede1_rhodamine (upper panels). These data reveal no substantial (i.e., >2-fold) linkage selectivity for the free UBA domain. The results from a single experiment are presented here; replicates in similar assays typically varied by <10%. Small, random residuals were observed for the free UBA domain binding to chains (lower panels).

FIG. 15 shows that the bivalency of GST-Ede1 UBA preferentially promotes binding to K63-polyUb over K48-polyUb. (A) The process of making the functionally monomeric version of GST-Ede1 (GSTfmm) from a mixture of GST-His (gray subunits) and GST-Ede1 (white subunits) is shown schematically. (B) GST subunit exchange can be seen by comparing the starting materials to the final GST-fmm mixture after separation by native PAGE. The heterodimer has an intermediate pI and migrates between the positions of the two homodimers. (C) GST-Ede1 and GST-fmm were used to pull-down ¹²⁵I-labeled Ub₄ chains. The upper panel shows an autoradiogram of SDS-PAGE-separated Ub₄ chains. To resolve GST-Ede1 and GST-His by SDS-PAGE, smaller amounts of the same samples were separated for a longer time on a second gel (lower panel). The amounts of GST-Ede1 and GST-fmm used in the assay were adjusted to contain similar amounts of the GST-Ede1 subunit (upper band). Separate negative controls (GST-His) were performed to account for different amounts of total protein on the GST-Ede1 and GST-fmm beads. The results of two assays are averaged in (D). (E) SPR analysis confirms this result. K63-Ub₄ affinity and selectivity were reduced with GST-fmm (upper panel) relative to the GST-Ede1 homodimer. Data shown are an average from two independent trials that differed by <10%. Deviations from the fits are shown in the lower panel.

FIG. 16 demonstrates that hHR23A-UBA1 is a K48-selective UBA domain. (A) Fluorescence anisotropy binding data for hHR23A-UBA1 interacting with K63-Ub4 or K48-Ub₄ indicate a preference for K48-polyUb. CSP mapping was used to identify the UBA1 surface responsible for binding to (B) monoUb and (C) K48-Ub₂. Upper panels show the amide CSPs as a function of ¹⁵N-UBA1 residue number. Residues that were significantly affected by binding (CSP Δδ>0.10 ppm, or signal attenuation >60%) are mapped to the UBA1 structure (Mueller et al., 2002) (1IFY.pdb) below (spheres). Blue spheres indicate the residues that were only perturbed upon K48-Ub₂ binding. UBA1 interacts with K48-Ub₂ with an expanded set of residues in a configuration that is similar to the linkage-specific binding of UBA₂ from the same protein. (D) To show this similarity, UBA1 (contacts indicated as before by spheres) was aligned to the UBA₂ coordinates from its bound complex with K48-Ub₂ (1ZO6.pdb) (Varadan et al., 2005). Ub₂ is in yellow ribbons: UBA₂ is in white ribbons; the aligned UBA1 is in dark gray ribbons. The putative interface of UBA1 with the distal Ub is similar to the monoUb interface (red spheres), whereas additional residues on the other side of UBA1 (blue spheres) that are specifically perturbed in the K48 interaction could make linkage-specific interactions with the isopeptide region and proximal Ub.

FIG. 17 shows that oligomeric UBA proteins may achieve K63-selectivity through linkage-specific avidity. (A) Domain maps for two UBA proteins, c-IAP2 (top) and p62/SQSTM1 (bottom), that are functionally linked to K63-polyUb binding; the UBAs and the oligomerizing domains are highlighted (dark gray). (B) In their oligomeric forms, these proteins may present arrays of UBAs that are specific for K63-polyUb.

FIG. 18 demonstrates that Ede1 residues E1364 and K1365 are not determinants of polyUb affinity or selectivity. SPR was used to assay Ub₂ binding by GST-fused Ede1(E1364A) and Ede1(K1365A). K63-Ub₂ (A) and K48-Ub₂ (B) data for these mutants are presented together with data for wild-type Ede1 for comparison. The fit values are shown in (C) and indicate that these residues do not contribute significantly to polyUb binding or selectivity.

FIG. 19 depicts Ede1 UBA interactions with monoUb and diUb detected by NMR CSPs. CSP plots for ¹⁵N-labeled (A) monoUb, (B) K63-Ub₂, and (C) and K48-Ub₂ titrated with Ede1 UBA show that a similar set of residues on the Ub surface is recognized in each interaction. Plots in (B) and (C) correspond to the structural maps in FIG. 13D-G.

FIG. 20 presents a model for avid binding of dimerized GST-UBA domains to K63-linked polyUb. A molecular model suggests that dimerized GST domains could bring their UBA fusion partners together closely enough in space to interact simultaneously with adjacent, K63-linked Ubs. This model was made by starting with the coordinates for a crystal structure of GST (1U87.pdb). The last structured residue on the terminus of the protein is T209, marked here in yellow spheres. The C termini in the dimer are about 28 Å apart. Typical GST-fusion proteins, like the UBA domains described herein cloned into pGEX-4T2 vectors, contain seven additional GST residues (FGGGDHP) and 10 or more vector derived residues (e.g., PKSDLVPRGS). It was assumed that both of these segments are relatively flexible. The coordinates from the structure of hHR23A were used to approximate a 17-residue, flexible linker followed by a UBA domain (here, UBA1 from hHR23A). When the resulting structure is compared to two K63-linked Ubs, it is clear that simultaneous, avid interactions with a single K63-Ub₂ molecule would be possible for two UBA domains brought close in space by their dimeric GST fusion partners. However, a structural basis for K63-vs. K48-polyUb linkage preference is not clear from this model.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Linkage-Specific Avidity Defines the Lysine 63-Linked Polyubiquitin-Binding Preference of Rap80

The inventors of the present invention suspected that some multivalent Ub-binding proteins may achieve linkage selectivity by exploiting the distinct orientation and spacing of Ub units that result from a particular polyUb linkage. Multiple ubiquitin-binding domains (UBDs) could be arrayed in space to optimize simultaneous interactions with both Ub units in a configuration characteristic of one type of Ub-Ub linkage, but not another. For the target polyUb linkage, binding by the first of multiple UBDs to one Ub would position the second UBD favorably for interaction with a nearby Ub in the chain. Because the second binding event occurs between binding partners at high local concentrations, it is potentially much more favorable. This forms the basis of linkage-specific polyUb recognition, as well as polyUb versus monoUb specificity. This type of cooperative binding is termed avidity; hence, the mechanism is called “linkage-specific avidity.”

The human protein Rap80 was examined as a potential model of linkage-specific avidity. At the site of DNA double-strand breaks in human cells, an early signaling cascade leads to the recruitment of the Ub E2 enzyme Ubc13 and the E3 RNF8, which then ubiquitinate one or more substrates at the site of the damage with K63-linked polyUb (Bennett and Harper, 2008; Kolas et al., 2007; Mailand et al., 2007; Wang and Elledge, 2007; Huen et al., 2007). Rap80 links these early events to proteins critical for repair by binding to the K63-polyUb signal with N-terminal tandem UIMs (tUIMs), whereas a central Rap80 domain binds an associated complex that includes Abraxas, the tumor suppressor BRCA1, and the deubiquitinating enzyme BRCC36 (Kim et al., 2007a; Sobhian et al., 2007; Wang et al., 2007; Wang and Elledge, 2007; Yan et al., 2007). The importance of the localization activity is evident from clinical BRCA1 mutants that do not assemble into the complex with Rap80 and thus fail to reach the sites of DNA damage (Sobhian et al., 2007).

Although UIM domains are not known to have a large linkage preference in isolation, Rap80 bound more K63-polyUb than K48-polyUb in GST pull-down experiments (Kim et al., 2007a; Sobhian et al., 2007). UIMs are 18-21 amino acids in length and are ideal for structure prediction and molecular modeling because of their simple, α-helical structure (Fisher et al., 2003; Hofmann and Falquet, 2001). Modeling was performed based on the structure of a UIM bound to monoUb and it was found that the 7-residue linker between the Rap80 tUIMs could position the domains ideally for simultaneous interactions with two K63-linked Ub units. In contrast, modeling based on a K48-diUb structure predicted that avid interaction with K48-diUb would be impossible or inefficient, requiring longer K48 chains for simultaneous contacts. Consistent with these models, the Rap80 UIM linker is shown to define selectivity by optimizing avid binding across a single K63 linkage. It is demonstrated herein that linkage-specific avidity also underlies the selective preference for K48-polyUb in a tUIM protein with a shorter linker, human ataxin-3. Using tUIMs as a model, the general principles that underlie linkage-specific avidity and polyUb affinity in multi-UBD proteins are established.

### Discussion

Linkage-Specific polyUb Binding. The paradigm for linkage-selective polyUb recognition has been shaped mostly by examples from the Ub-associated (UBA) domains. The molecular basis for K48-selective binding has been described for UBA2 from human hHR23A (Varadan et al., 2005) and for UBA in Mud1 from fission yeast (Trempe et al., 2005). In these cases, all of the elements that confer selectivity are contained within a single UBA domain, and specific recognition of K48-polyUb is achieved through binding at an interface centered on the Ub-Ub isopeptide bond. Recently, a study by Lo et al. (2009) revealed how the CC2-LZ domain of NEMO binds linear and K63-linked diubiquitin. Both halves of the dimeric CC2-LZ domain engage linear and K63-linked diubiquitin along slightly different extended surfaces that include the Ub-Ub junction.

Though selective recognition of polyUb chains by single domains is likely an important mechanism, multiple UBDs are commonly found in Ub-binding proteins and protein complexes (Hicke et al., 2005; Reyes-Turcu et al., 2008). In these cases, large cooperative binding effects may make the individual domain interactions less relevant. For example, multivalency is thought to underlie the physiologically relevant affinities achieved at the endosome when Ub receptors bind oligomerized, multimonoubiquitinated, or polyubiquitinated cargoes with a series of tandem UBDs that, in isolation, each bind Ub poorly (Barriere et al., 2006; Haglund et al., 2003; Hawryluk et al., 2006; Hicke and Dunn, 2003). As described herein, it has been demonstrated that nonselective or weakly linkage-selective single UIMs can also achieve considerable linkage specificity when the arrangement of the domains makes avid binding to one polyUb topology more favorable than others.

The model described herein differs from the isopeptide-centered recognition in the examples above because linkage-specific avidity requires no specific contacts at or near the isopeptide bond. It remains to be determined whether UBDs other than UIMs can exert a linkage preference through avidity. A role for linkage specific avidity in specific binding for other polyUb topologies or for Ub-like protein polymers such as polySUMO (Kerscher et al., 2006) is also speculative. Nonetheless, by using tUIMs as a model, some of the general principles that should govern these multivalent interactions has been established. Avid binding is highly sensitive to the orientation of the binding units with respect to each other and the flexibility between the units.

Systems with less flexibility are expected to have higher affinities and specificities because entropic costs are higher when more flexible linkers need to bring binding units together in space and because flexible linkers do not restrict binding units to a small set of potentially distinguishing conformations. Also, the intrinsic affinities of individual domains contribute to specificity and affinity; because avidity, at best, only multiplies receptor affinities, weak-binding receptors will gain less from a similar multivalent arrangement than tight ones (Bobrovnik, 2007; Schleif and Wolberger, 2004). Potentially, UBDs distant in primary sequence, or even on separate polypeptides, could achieve linkage selectivity through avid interactions so long as flexibility between properly positioned domains is minimized.

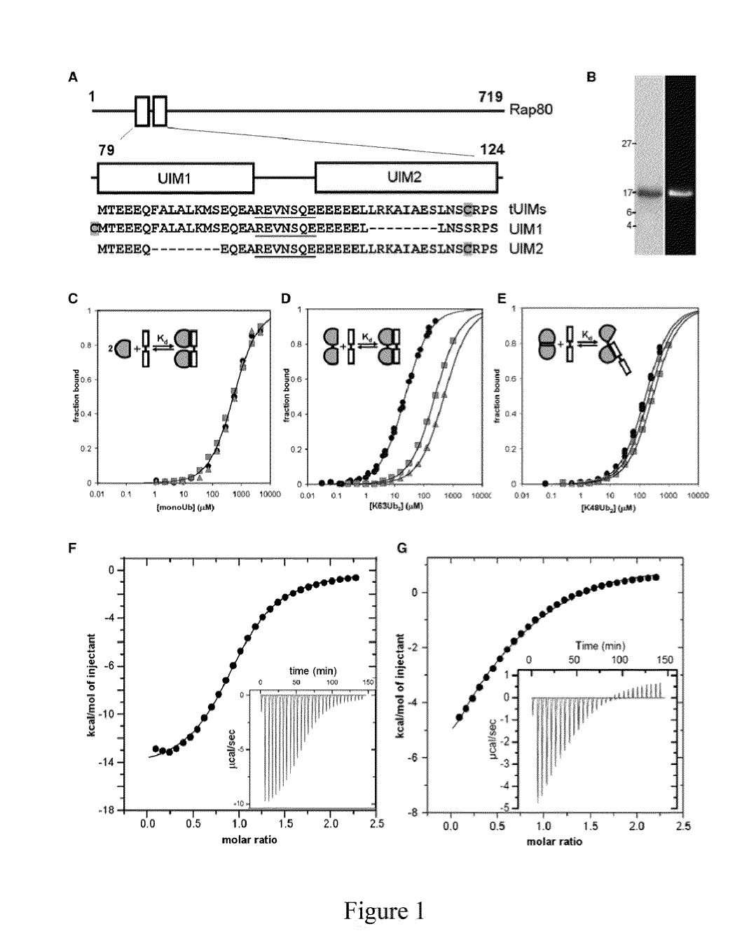

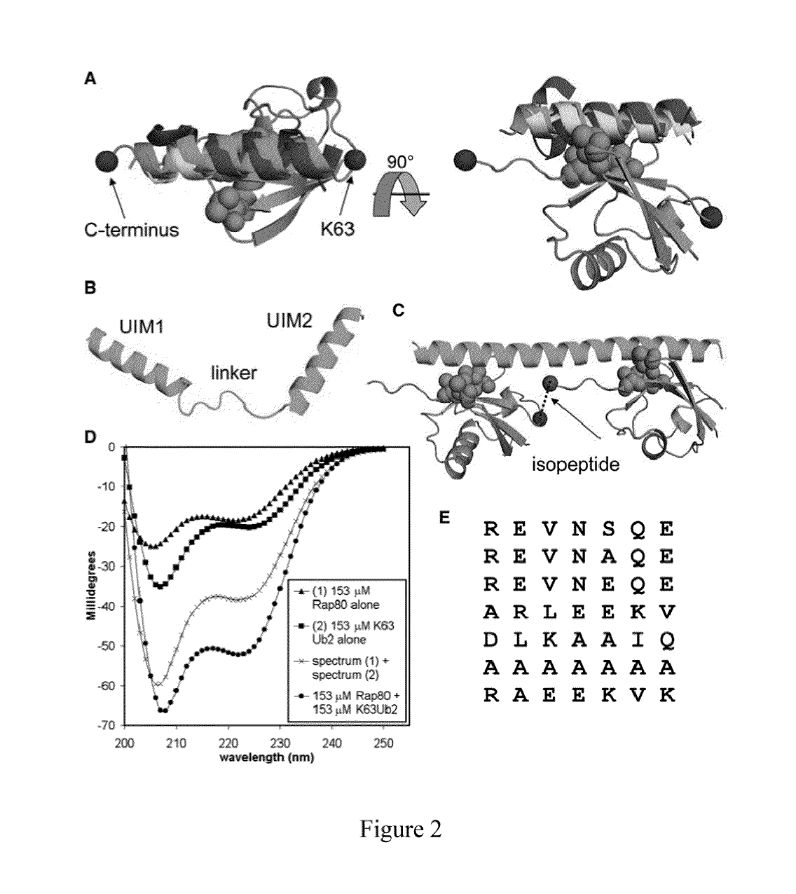

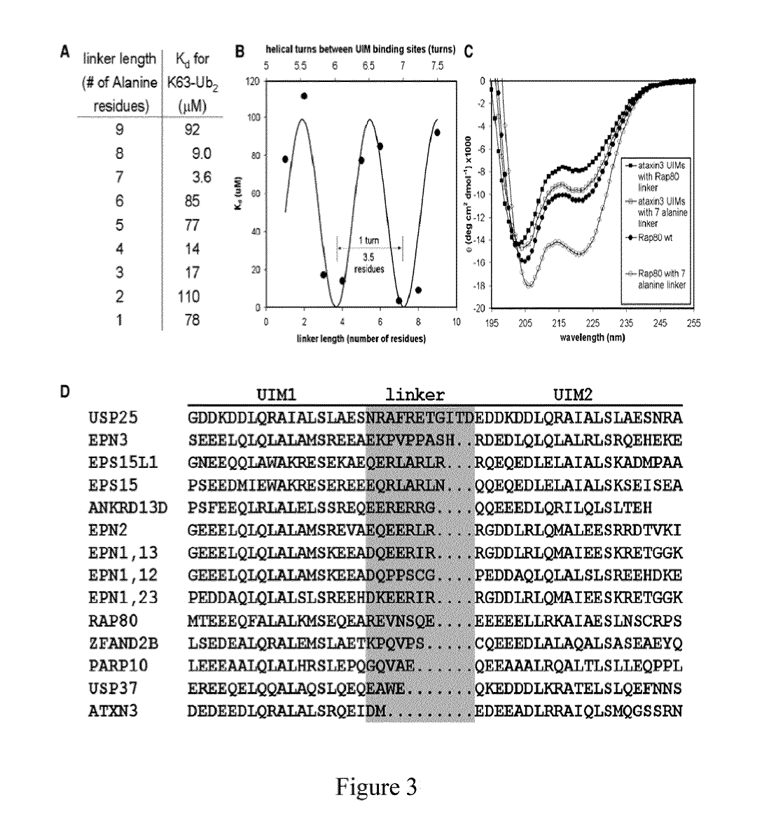

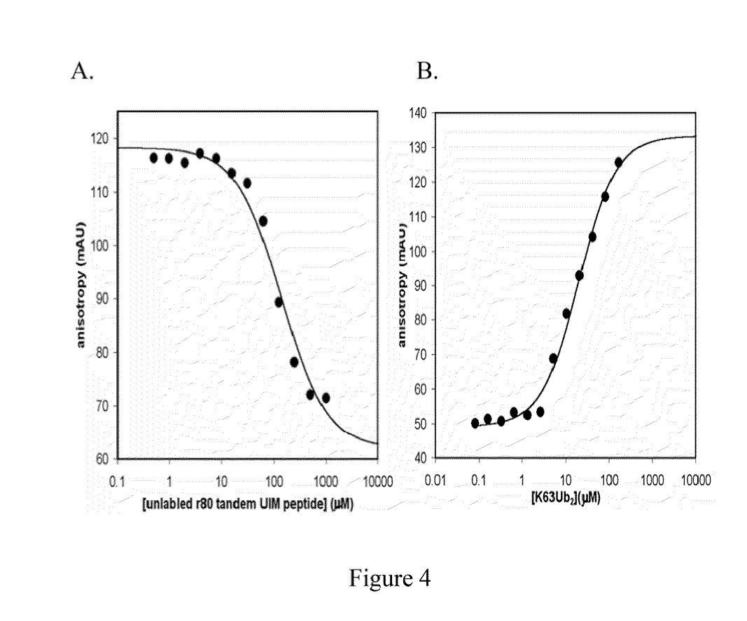

Protein Function and Linkage-Specific Avidity. As shown herein, the sequence linking UIM domains can be the major determinant of K63 or K48 linkage preference. Among human proteins with close tUIMs, the largest group has linkers similar to the Rap80 length (i.e., 7 or 8 residues), which, in our studies, support K63-specific binding (FIG. 3D). It will be interesting to see whether future studies bear out this predicted relationship to K63-polyUb.

Though a K48 preference for ataxin-3 was measured, there is evidence for a preference to depolymerize mixed K63/K48-linked polymers (Winborn et al., 2008). Mixed linkage chains have only recently been studied functionally, and their structures and basis for their recognition are unknown (Ikeda and Dikic, 2008; Kim et al., 2007b). It is noted that, because mixed chains contain more than one type of isopeptide bond, specific recognition could not be achieved by a single isopeptide-directed interaction; instead, multivalent interactions across more than one Ub unit may be required. Careful binding studies are needed to understand mixed chain recognition by, for example, the ataxin-3 tUIMs.

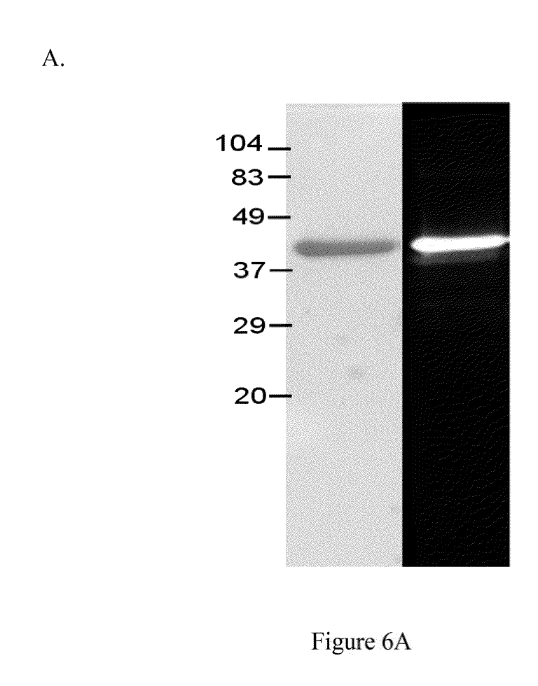

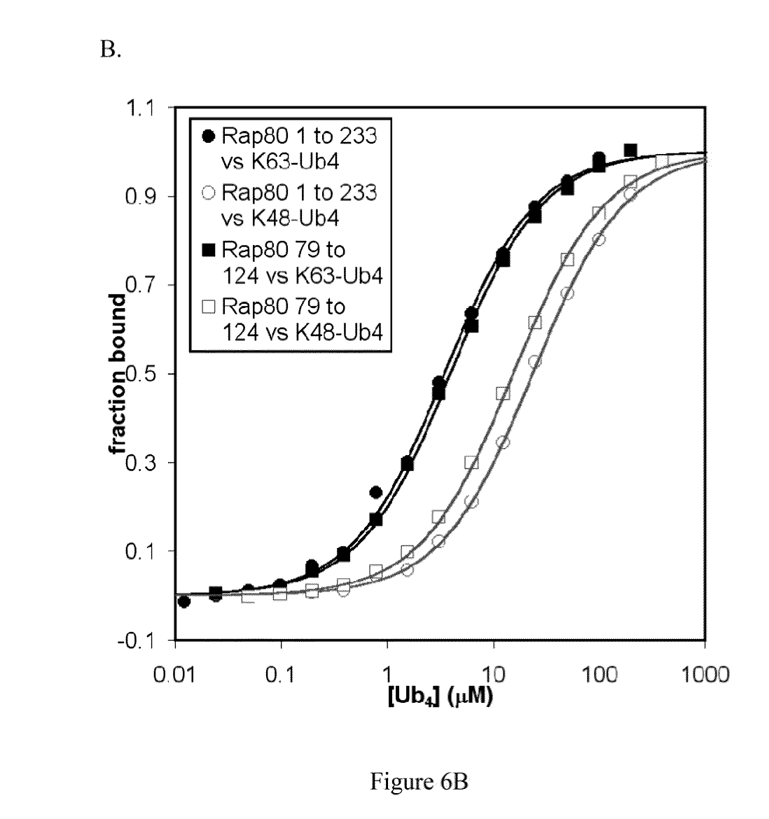

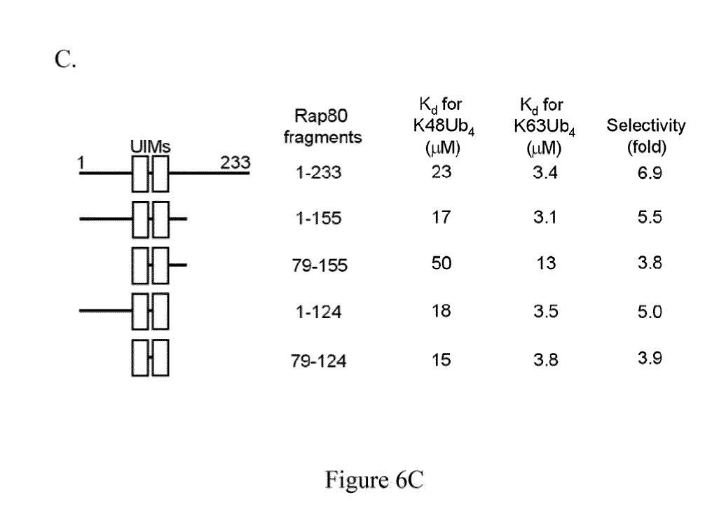

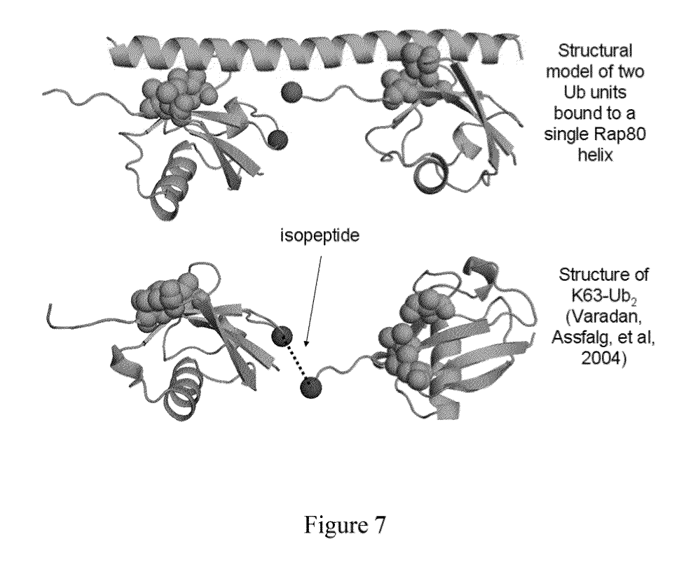

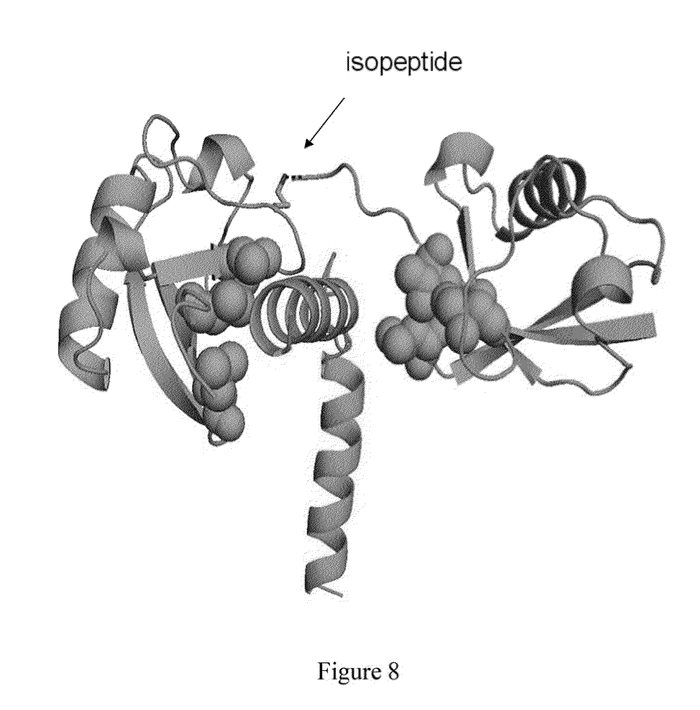

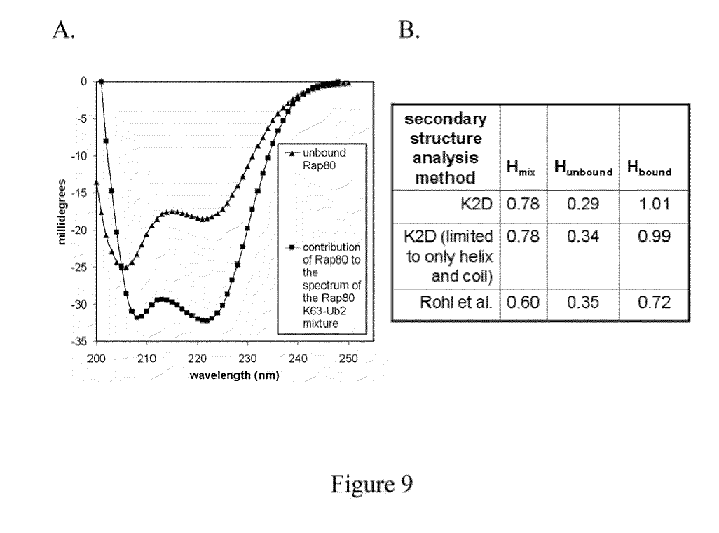

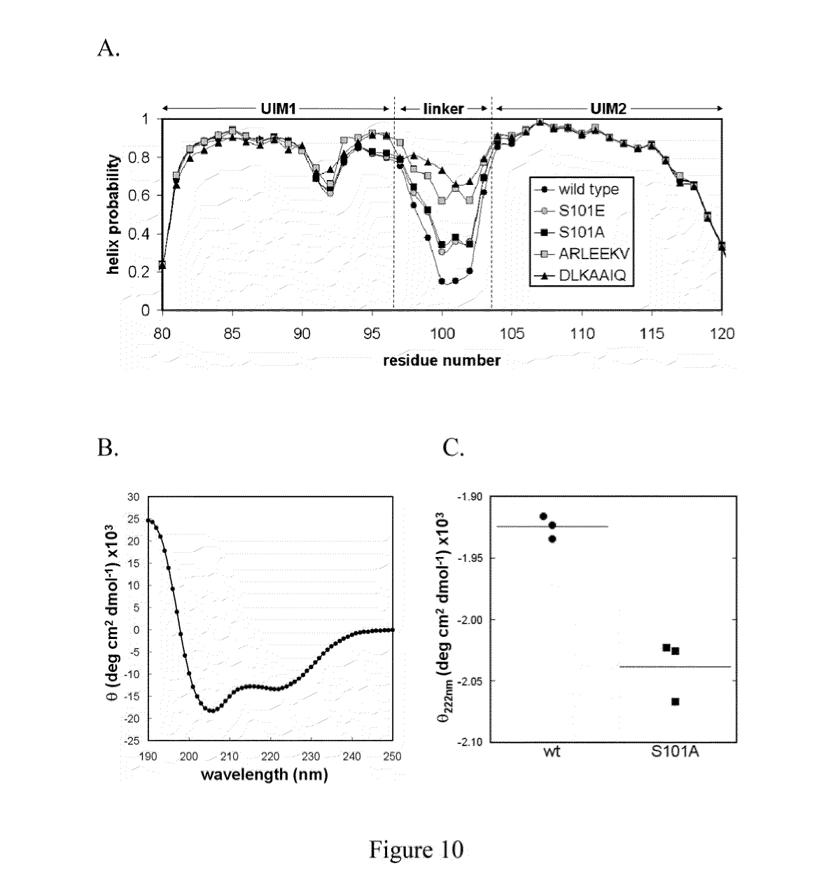

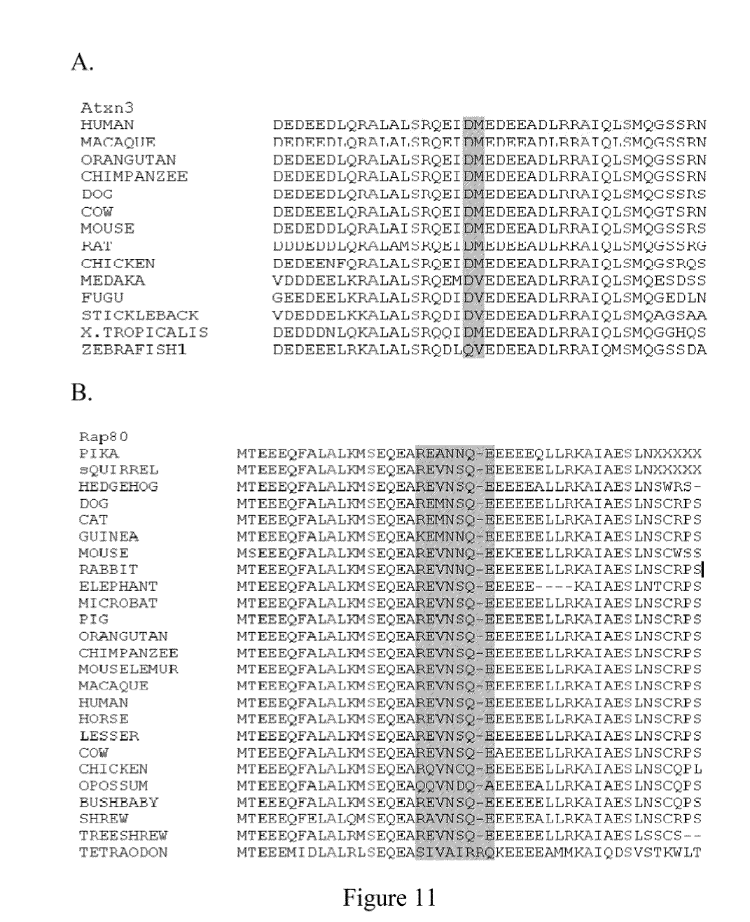

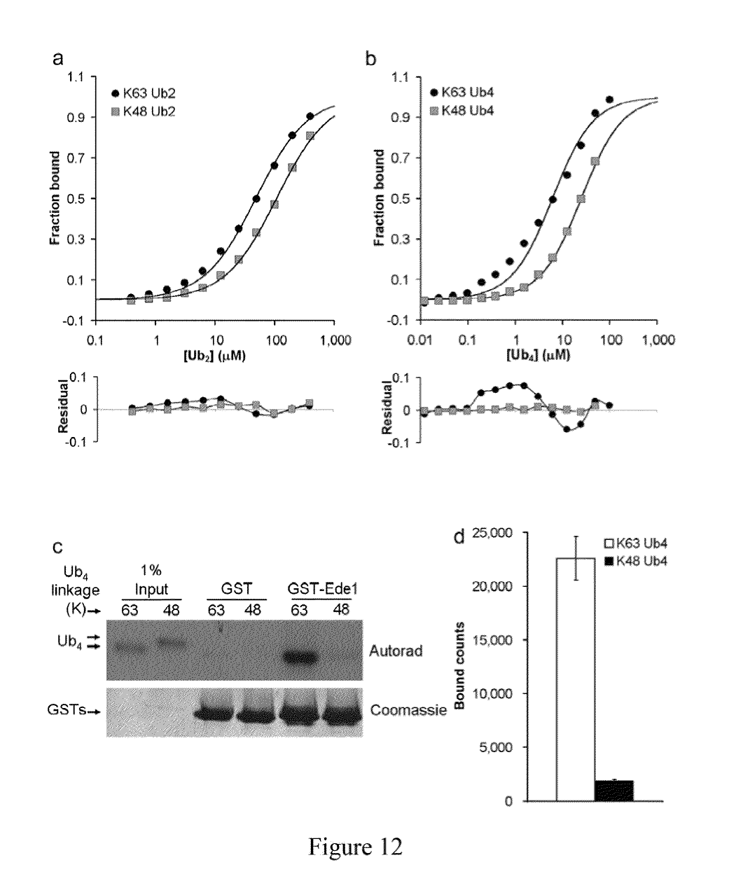

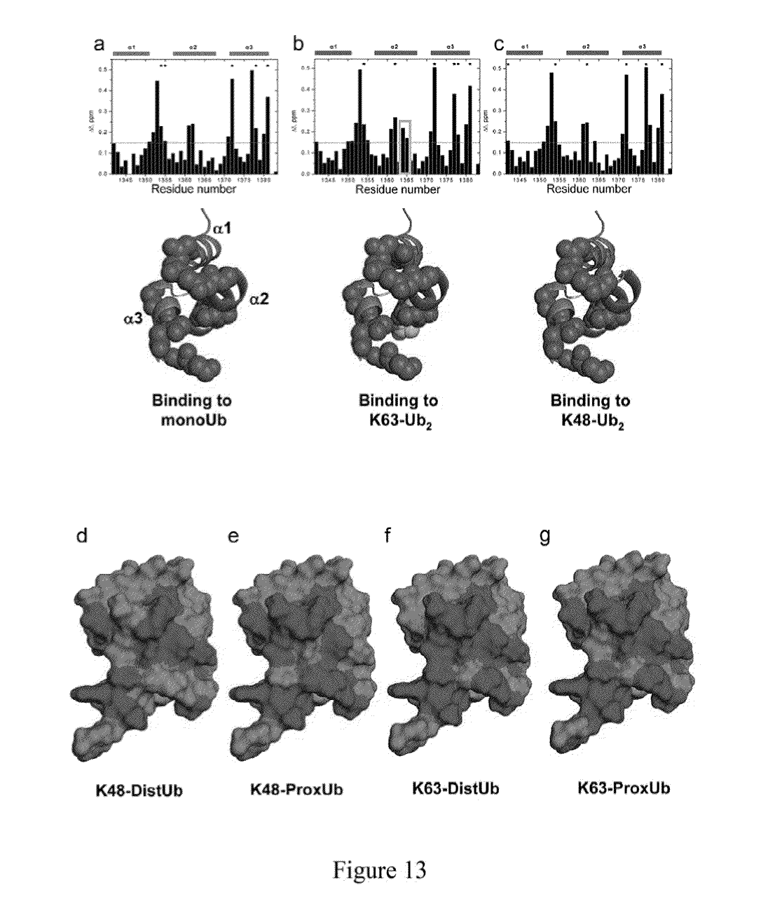

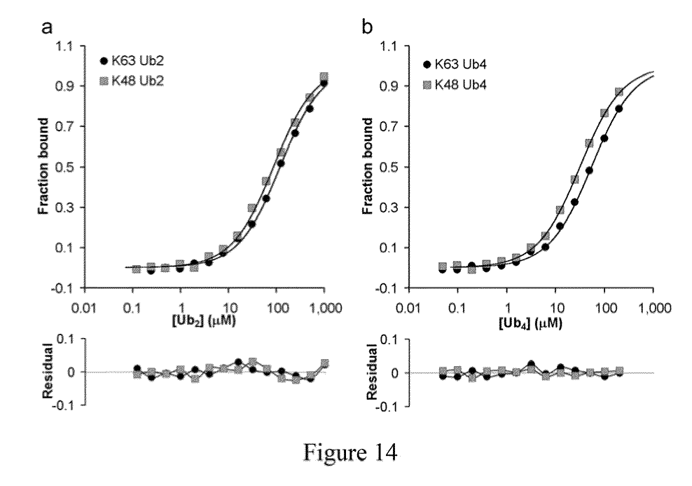

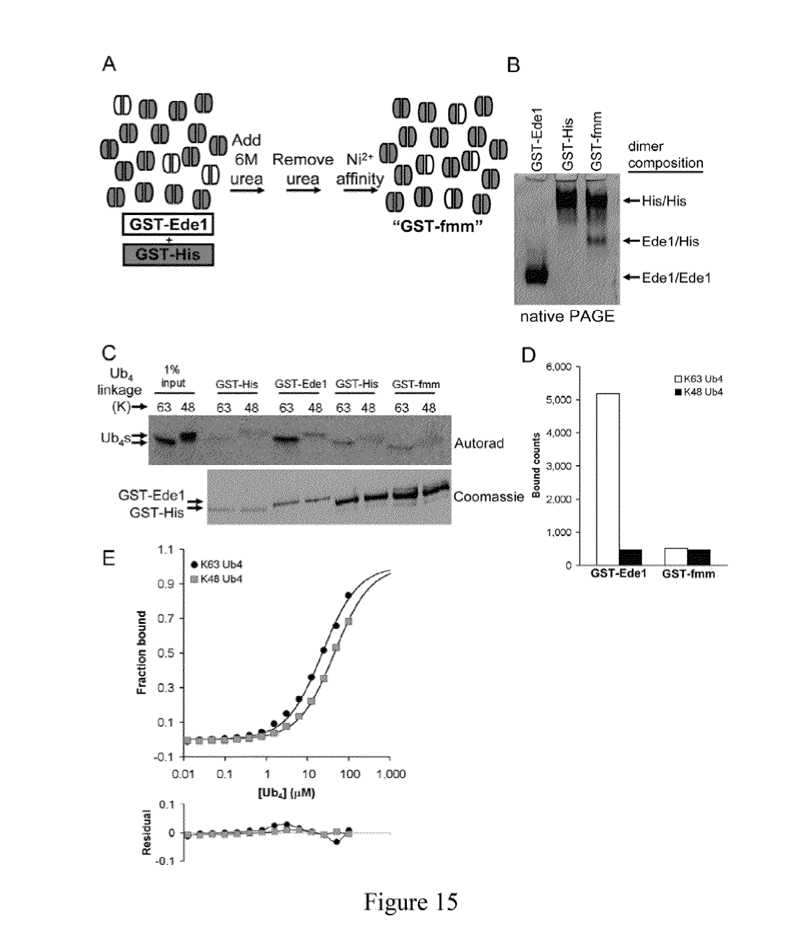

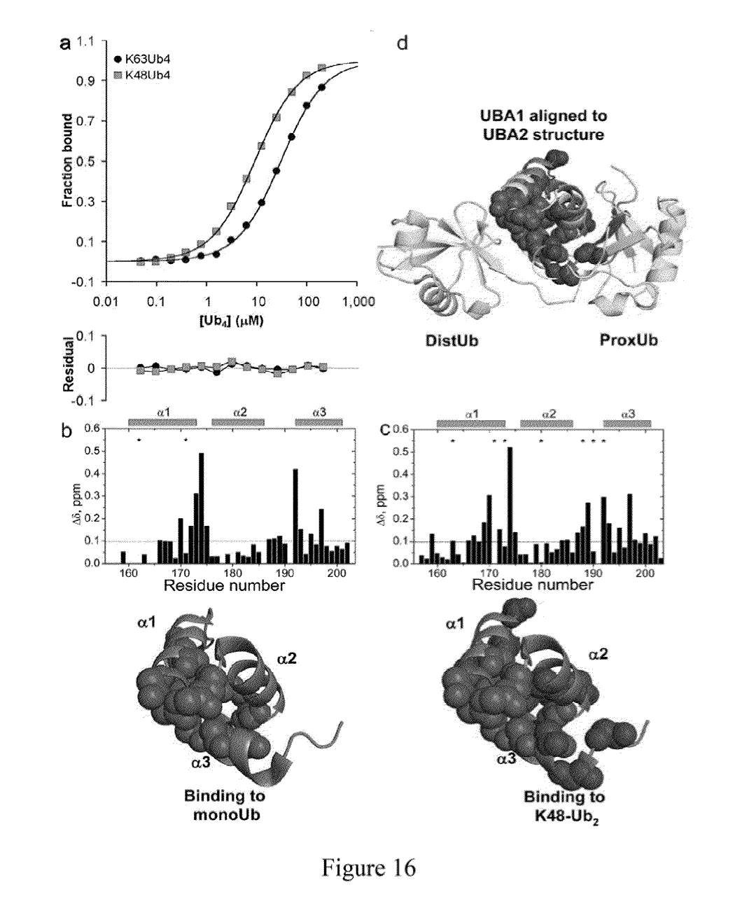

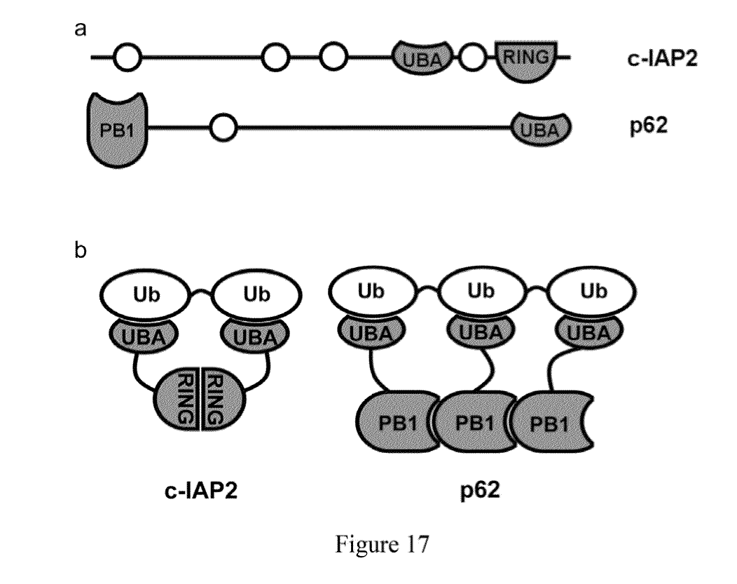

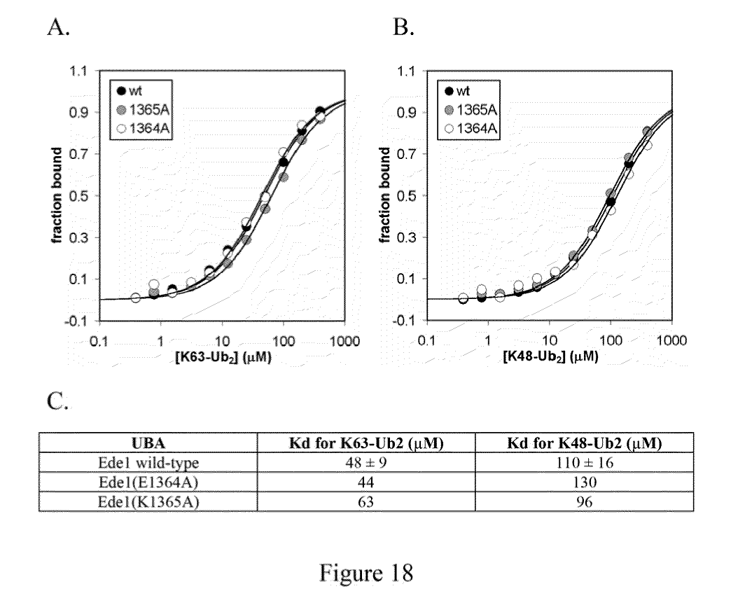

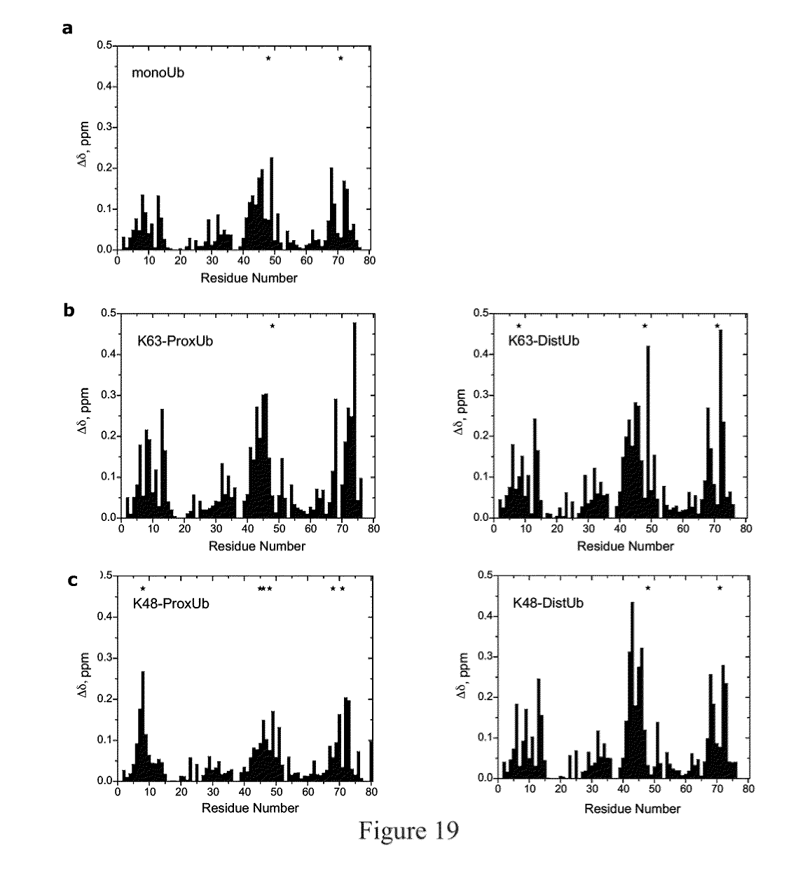

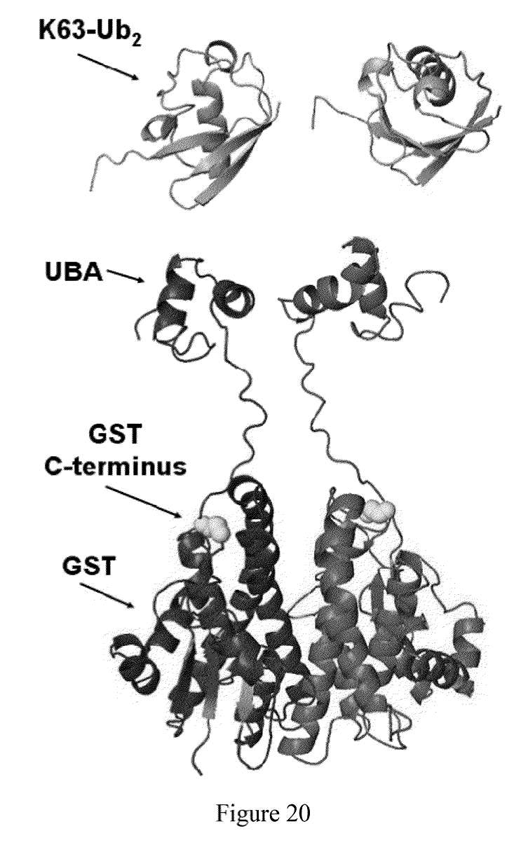

The solution-state measurements with a soluble fragment of Rap80 (residues 1-233) longer than the minimal tUIMs indicate only a 7-fold preference for K63-Ub₄ over K48-Ub₄ (FIG. 6). Although published results indicate an apparently large selectivity for long K63-linked chains over K48-linked chains by Rap80 fragments (Kim et al., 2007a; Sobhian et al., 2007), it is important to recognize that these studies used immobilized, GST-fused versions of Rap80 to pull down polyUb chains and qualitative immunoblotting to assess the binding. Because avidity defines Rap80's linkage preference, artificial valencies introduced though GST dimerization and immobilization could have a profound impact on the results. One pull-down result also suggested that tight binding to K6-linked polyUb is possible (Sobhian et al., 2007). Because BRCA1, an indirect binding partner of Rap80, is an E3 ligase that can make K6-linked chains in vitro, the possibility of an interaction with K6 is provocative. However, several lines of evidence suggest that the in vivo binding partner for the Rap80 UIMs is a K63-linked polyUb chain (Huen et al., 2007; Kolas et al., 2007; Mailand et al., 2007; Plans et al., 2006; Wang and Elledge, 2007; Yan and Jetten, 2008), and in vivo roles for K6 chains have not been established.

Rap80 is phosphorylated on multiple serine residues by the ATM kinase in response to DNA damage. Serine 101 in the tUIM linker is one of these sites, although its phosphorylation is not required for efficient localization to DNA damage foci (Kim et al., 2007a; Sobhian et al., 2007). Nonetheless, in light of the results that the nature of the linker can affect polyUb binding, it was questioned whether this modification could drive a structural transition in the linking sequence that could activate K63-polyUb binding. Although a phosphopeptide suitable for binding studies was not produced, the series of linker variants that were tested suggests that S101 phosphorylation is unlikely to promote the relatively large structural transition required to dramatically affect affinity and selectivity (see S101E in Table 2). However, other modifications may indeed be used to regulate tUIM affinity. A recent study shows that residues in and around the tUIMs of USP25 (FIG. 3D) are SUMOylated and that this modification regulates polyUb binding and enzymatic activity of the enzyme (Meulmeester et al., 2008).

To date, the functional significance of linkage specificity has not been demonstrated for any polyUb receptor (Kim and Rao, 2006). Whereas the Rap80 UIMs achieve specificity that is comparable in magnitude to other linkage-selective proteins, the somewhat modest preferences of these proteins (typically less than 10-fold) may call into question the functional role of linkage preference. In vivo studies are required to determine the physiological significance of linkage specificity for Rap80 and other polyUb-binding proteins.

### II. Avid Interactions Underlie the K63-Linked Polyubiquitin Binding Specificities Observed for UBA Domains

A key finding in support of the model that diverse polyUb binding preference should exist among Ub receptor proteins to promote the proper downstream consequences was that a large class of ubiquitin binding domains known as ubiquitin associated (UBA) domains contains a diverse set of ubiquitin specificities (Raasi et al., 2005). Glutathione-S-transferase (GST) fusions of UBA domains from more than 30 proteins, including all but one of the UBAs from budding yeast, were evaluated by quantitative pull-down assays for mono- and polyUb binding preferences. K48- and K63-polyUb selectivities were observed, as well as tight binding to monoUb that was associated with little polyUb linkage preference. Although K48-specific UBAs were known (Raasi et al., 2003), this was the first report of K63-linkage selectivity for isolated UBA domains. This study indicated that UBAs could present a diverse range of linkage-specific epitopes, and that linkage selectivity was achieved mainly at the level of these small, modular domains.

It was expected that the reported K63-specific UBA interactions would be explained by binding at a linkage-specific epitope on K63-polyUb, which the present study was designed to identify. As described herein, the apparent K63-selectivity of some UBAs is actually due to avid interactions that are artificially promoted in the dimeric GST-fusions used to classify the domains. UBAs formerly considered K63-selective based on the GST-UBA fusions lose or reverse selectivity as free domains. Accordingly, those domains individually exhibit no K63-selective contacts with polyUb. Previous studies of UBAs are re-examined in light of this linkage preference artifact to resolve some functional and mechanistic inconsistencies. How this artifact suggests an additional level of linkage specificity that could arise from multivalent arrangements of UBA domains in nature was also examined.

### Discussion

A widely cited conclusion is that isolated, minimal UBA domains contain a broad range of intrinsic Ub binding specificities, including K48- and K63-polyUb preference (Raasi et al., 2005). As shown herein, the assays used to reach those conclusions have artificially promoted K63-polyUb binding for some UBA domains, and thus may have overestimated the range of UBA•polyUb specificities attributable to the minimal UBA domain. Accordingly, no structural or biophysical evidence was seen for K63-selective binding in isolated, free UBA domains. Of the seven UBA domains originally identified as K63-selective, it was been shown that one is non-selective (Ede1 UBA from yeast) and two are actually K48-selective in isolation (human hHR23A UBA1 and yeast Ubc1). By extension, it is expected that the homologs of these UBAs, yeast Rad23 UBA1 and human E2-25k UBA, respectively, are also K48-selective in isolation. The remaining two domains, both from the Arabidopsis protein DRM2, have not been examined.

The apparent source of the GST-fusion artifact that has been observed is a type of linkage specific avidity in which the dimeric GST moiety can position two UBAs close in space to make simultaneous contacts with K63-polyUb, but not K48-polyUb. As shown herein, closely-spaced tandem UIM domains can achieve polyUb linkage preference through the same mechanism. This work extends the range of configurations that can result in linkage specific avidity, as well as the types of UBDs that can be involved.

The question arises as to why should some multivalent arrangements promote binding to a K63-polyUb chain over a K48-polyUb chain that has an equivalent number of Ub binding sites. As has been shown herein for tandem UIM domains, the orientation and spacing of two UBDs can promote avid binding to one linkage, but not another, and thus provide an effective means of linkage selectivity. Considering the flexibility that probably exists in the GST-UBA linking sequence of the constructs examined (FIG. 20), it seems unlikely that the GST and linker alone could exert much influence over the orientation and spacing of UBA fusions. However, as mentioned above, UBA self-association in the context of a dimeric GST-fusion could provide control over the orientation of the UBAs. Alternatively, the different conformations of K48- and K63-polyUb chains in solution (Ryabov et al., 2006; Varadan et al., 2004) could either promote or restrict access to avid binding modes by multiple UBAs.

Pull-down assays with immobilized Ub-binding proteins are widely applied to assess linkage specificity, particularly since small amounts of K48- and K63-polyUb have been made commercially available. The results herein suggest that any immobilization of UBD proteins on a solid surface such as glutathione-coated beads or SPR chips may result in artificial multivalency that can profoundly influence polyUb binding properties such as chain length preference, linkage preference, and affinity. Multivalent interactions allow individual sites to re-bind after dissociation and therefore slow observed off-rates; non-equilibrium wash steps in pull-down assays can exaggerate these differences in off-rates, particularly for weak receptors. In fact, for typically weak Ub receptors, most of the retained polyUb chains in a pull-down assay are probably bound avidly. Thus, conclusions about intrinsic linkage preference drawn from such experiments should be re-examined. Another complication is that, to conserve chains, polyUb pull-downs often employ (typically) non-linear western-blots to achieve sensitive chain detection, and use anti-Ub antibodies that can differentially stain polyUb chains of different linkages. As well as being an additional source of error, western-blotting can also have the effect of reducing subtle differences in linkage preference to all or nothing conclusions.

The SPR assay is closer to an equilibrium binding measurement, but because immobilization is achieved with divalent anti-GST antibodies, the commonly used GST-fusion based version of this assay may add a double layer of valency. Indeed, it seems that all traces of multivalency could not be eliminated from the SPR assay (FIG. 15E). Ideally, polyUb binding studies would be performed in the solution phase with full-length proteins near their physiological concentrations, or in the context of a relevant protein complex. Since this standard is impractical or impossible for most polyUb receptors, a reasonable compromise may be to at least avoid a known source of experimental artifact (GST fusion or immobilization) in favor of solution-based assays with a UBD or UBD-protein to determine linkage selectivity.

Avidity artifacts in previous polyUb binding studies may have led to some confusion about the functional significance of polyUb selectivity in UBD proteins (Kim, 2006). Human hHR23A UBA1 and its yeast homolog were originally classified8 as K63-selective but occur in proteasomal ubiquitin receptor proteins, where K48-polyUb binding is presumed to be more relevant. Likewise, two other members of the original K63-selective class, yeast Ubc1 and its human homolog E2-25K, have stronger functional connections to K48-polyUb pathways. Consistent with the finding of K48-selectivity, both are involved in endoplasmic reticulum-associated degradation (ERAD), a pathway that requires K48-polyUb31, and E2-25K assembles K48-linked chains exclusively in vitro (Chen et al., 1990). In fact, the earlier study (Raasi et al., 2005) identified just one K48-selective UBA in yeast (UBA₂ from Rad23), although K48-linked chains are likely the most common type of polyUb (Xu et al., 2009). The present study resolves these inconsistencies and recognizes the intrinsic K48-polyUb selectivity of many more UBA domains. Nonetheless, conclusions about the role of these relatively modest linkage preferences will require studies that directly examine the link between selectivity and function.

Previous work has explained intrinsic K48-polyUb linkage-selective binding by human hHR23A UBA₂13; and fission yeast Mud1 UBA11. These UBAs meet the expectations for intrinsically linkage-selective domains because they present similar, K48-specific epitopes on their surfaces. UBA₂ binds K48-Ub₂ at a contiguous interface that includes the isopeptide bond and both Ub hydrophobic patches, the sites of all known UBA•Ub interactions. The present study indicates a similar arrangement for UBA1 of hHR23A interacting with K48-Ub₂. This is because K48 is adjacent to the hydrophobic patch, and K48-Ub₂ adopts a structure that brings both hydrophobic patches into close proximity (Varadan et al., 2002). However, it is unclear how the small UBA domain could achieve an analogous, linkage-specific interface with K63-Ub₂ because lysine 63 is not close to the hydrophobic patch, and K63-linked ubiquitins adopt an elongated, open structure in solution (Varadan et al., 2004). Intrinsic K63 selectivity, at least in the mold of UBA₂•K48-Ub₂ recognition, may not exist among UBA domains or any of the other small UBDs that require contacts with the Ub hydrophobic patch (e.g., CUE, UIM, or NZF domains). In contrast, larger and more extended UBDs appear to be capable of intrinsic K63-polyUb selectivity, as recently shown for the CC2-LZ domain of NEMO34. CC2-LZ engages linear or K63-linked diUb along an extended surface that includes both Ub units and the junction between them.

If the range of signaling functions accomplished by UBA proteins requires a similarly diverse range of polyUb linkage preferences, the work described herein indicates that the origins of linkage selectivity are more complex than the intrinsic specificities of minimal domains. The GST effect described here suggests how UBA proteins can use two mechanisms to diversify polyUb linkage preferences: some UBAs are intrinsically K48-specific, and K63-polyUb selectivity can arise from certain avid combinations of intrinsically non-specific UBA interactions. Unfortunately, few measurements of UBA protein binding specificity have considered the influence of multiple domains in a complex or the oligomeric state of a single-UBA protein. Nonetheless, a survey of the literature yields several cases where oligomeric proteins that contain UBA domains achieve K63-selectivity. One example is the IAP (inhibitor of apoptosis) proteins, a family of anti-apoptotic proteins that are involved in NF-κB signalling, in which UBA-mediated IAP interaction with K63-linked polyUb is critical for function (Broemer et al., 2009). One recent study showed that c-IAP2 is K63-selective and that polyUb binding required not only the UBA domain, but also an adjacent, dimerizing RING domain (FIG. 17) (Gyrd-Hansen et al., 2008). The c-IAP2 RING domain may determine the selectivity for polyUb in the same way that GST modulates selectivity of GST-Ede1 UBA, i.e., through linkage-specific avidity.

In another example, the highly oligomeric p62/SQSTM1 is a multi-functional scaffolding protein with links to K63-polyUb signaling in NF-κB and autophagy pathways (FIG. 17) Seibenhener et al., 2007; Tan et al., 2007; and Bjorkoy et al., 2006). Solution-phase measurements of linkage selectivity have not been published for p62 UBA, though there is some evidence that the isolated domain binds Ub weakly and without regard to linkage (Raasi et al., 2005). One study showed that full-length p62 binds K63-polyUb preferentially in vivo (Seibenheneer et al., 2004). In another (Bjorkoy et al., 2005), localization to autophagosomes, also likely to be signaled by K63-polyUb (Olzmann et al., 2008; Tan et al., 2008; and Tan et al., 2007), was abrogated by point mutations in the PB1 domain that prevented p62 self-association as well as point mutations in the UBA domain that eliminated Ub binding. Interestingly, another oligomeric protein with a similar architecture to p62, NBR1, was recently shown to localize to sites of autophagy, though the role of oligomerization in Ub binding is less clear (Kirkin et al., 2009).

With regard to the Ede1 UBA, it is noted that yeast Ede1 may be effectively oligomerized when a group of endocytic network proteins including Ede1 gather at high density around ubiquitinated cargo to recruit oligomerized clathrin to the sites of endocytosis (Maldonado-Baez et al., 2008; Aguilar et al., 2003; and Gagny et al., 2000), a process that in some cases may involve K63-polyUb. Careful biophysical and structural studies will be required to determine whether the linkage-specific avidity observed for some artificially oligomerized UBA domains relates to a functionally relevant mechanism of K63-selective binding by UBA proteins.

Without further elaboration, it is believed that one skilled in the art, using the description herein, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Linkage-Specific Avidity Defines the Lysine 63-Linked Polyubiquitin-Binding Preference of Rap80

Linkage-specific polyubiquitin recognition is thought to make possible the diverse set of functional outcomes associated with ubiquitination. Thus far, mechanistic insight into this selectivity has been largely limited to single domains that preferentially bind to lysine 48-linked polyubiquitin (K48-polyUb) in isolation. A mechanism is proposed herein, linkage-specific avidity, in which multiple ubiquitin-binding domains are arranged in space so that simultaneous, high-affinity interactions are optimum with one polyUb linkage but unfavorable or impossible with other polyUb topologies and monoUb. The model used herein is human Rap80, which contains tandem ubiquitin interacting motifs (UIMs) that bind to K63-polyUb at DNA doublestrand breaks. The sequence between the Rap80 UIMs positions the domains for efficient avid binding across a single K63 linkage, thus defining selectivity. K48-specific avidity is also demonstrated in a different protein, ataxin-3. Using tandem UIMs, the general principles governing polyUb linkage selectivity and affinity in multivalent ubiquitin receptors are established.

### Materials and Methods

Plasmids and Proteins. All tandem UIM peptides were cloned in-frame between the Nde1 and BamHI sites in pET28a (Novagen) using a version of the vector in which the second amino acid (G) was mutated to P. The wild-type Rap80 peptide gene was amplified from human genomic DNA; the Vps27 sequence was amplified from yeast genomic DNA; the ataxin-3 sequence was subcloned from a plasmid previously described (Chad, Berke, et al., 2004). Table 1 contains a list of the peptide sequences used. Linker variations were introduced by PCR. All UIM peptides and proteins were expressed in E. coli and purified on Ni NTA agarose (QIAGEN) following the manufacturer's instructions, followed by gel filtration (Superdex 75 or Superdex 200) or anion exchange (MonoQ) chromatography when needed. MALDI-TOF mass spectrometry confirmed the expected molecular weights of the purified peptides and complete cleavage of the initiating methionine. Ub and K48- and K63-linked diUb and tetraUb were made as described (Raasi, and Pickart, 2005); for the K48 and K63 chains, the proximal Ub blocking residue D77 was left in place, and the distal Ub contained a single lysine-to-arginine substitution at position 48 or 63, respectively.

TABLE 1
Tandem UIM Peptide Sequences

| Tandem UIM | |
|---|---|
| Peptide | Sequence |
| Rap80 tandem | (M)PSSHHHHHHSSGLVPRGSHMTEEEQFA |
| UIMs (wt) | LALKMSEQEAREVNSQEEEEEELLRKAIAE |
| | SLNSCRPS |
| Rap80 UIM1 | (M)PSSHHHHHHSSGLVPRGSHMTEEEQFA |
| | LALKMSEQEAREVNSQEEEEEELLNSCRPS |
| Rap80 UIM2 | (M)PSSHHHHHHSSGLVPRGSHMTEEEQEQ |
| | EAREVNSQEEEEEELLRKAIAESLNSCRPS |
| Rap80_2xlinker | (M)PSSHHHHHHSSGLVPRGSHMTEEEQFA |
| | LALKMSEQEAREVNSQEREVNSQEEEEEEL |
| | LRKAIAESLNSCRPS |
| Vps27 (wt) | (M)PSSHHHHHHSSGLVPRGSHMDRDYSTP |
| | EDEEELIRKAIELSLKESRNSASSEPIVPV |
| | VESKNEVKRQEIEEEEDPDLKAAIQESLRE |
| | AEEAKLRSERQKAC |
| Vps27 | (M)PSSHHHHHHSSGLVPRGSHMEEEEDPD |
| 7aa_linker | LKAAIQESLREARAEEKVKEDEEELIRKAI |
| | ELSLKESRNCA* |
| Rap80_6aa_linker | (M)PSSHHHHHHSSGLVPRGSHMTEEEQFA |
| | LALKMSEQEAREVAAEEEEEELLRKAIAES |
| | LNSCRPS |
| Rap80_nolinker | (M)PSSHHHHHHSSGLVPRGSHMTEEEQFA |
| | LALKMSEQEAEEEEELLRKAIAESLNSCRP |
| | S |
| Rap80_S101E | (M)PSSHHHHHHSSGLVPRGSHMTEEEQFA |
| | LALKMSEQEAREVNEQEEEEEELLRKAIAE |
| | SLNSCRPS |
| Rap80_S101A | (M)PSSHHHHHHSSGLVPRGSHMTEEEQFA |
| | LALKMSEQEAREVNAQEEEEEELLRKAIAE |
| | SLNSCRPS |
| Rap80_DLKAAIQ | (M)PSSHHHHHHSSGLVPRGSHMTEEEQFA |
| | LALKMSEQEADLKAAIQEEEEELLRKAIAE |
| | SLNSCRPS |
| Rap80_ARLEEKV | (M)PSSHHHHHHSSGLVPRGSHMTEEEQFA |
| | LALKMSEQEAARLEEKVEEEEELLRKAIAE |
| | SLNSCRPS |
| Ataxin3 (wt) | (M)PSSHHHHHHSSGLVPRGSHMDEDEEDL |
| | QRALALSRQEIDMEDEEADLRRAIQLSMQG |
| | SSRNC |
| Rap80 ataxin | (M)PSSHHHHHHSSGLVPRGSHMTEEEQFA |
| linker | LALKMSEQEADMEEEEELLRKAIAESLNSC |
| | RPS |

Fluorescent Labeling. Rap80 tUIM peptides were fluorescently-labeled on the sulfhydryl group of C121 (see FIG. 1). For longer Rap80 constructs, all cysteines except C121 were mutated to serine for site-specific labeling. Ataxin-3 and Vps27 UIM peptides were labeled on a single cysteine introduced at the C-terminus. Fluorescein-5-maleimide (Pierce) was coupled to cysteines following the manufacturer's instructions. After desalting through Sephadex G-25 resin, fluorescein-labeled tUIM peptides were separated from unlabeled peptides by anion-exchange chromatography (MonoQ column, GE Biosciences) by elution with a NaCl gradient in 50 mM Tris buffer, pH 7.9. The labeled peptides were essentially 100% pure by SDS-PAGE and Coomassie blue staining, and MALDI-TOF mass spectrometry confirmed the addition of a single fluorescein moiety.

Fluorescence Anisotropy Binding Assays. Fluorescence anisotropy measurements were made using a Fluoromax 4 fluorometer in L format, thermostatted at 25° C. Excitation and emission monochromators were set at 492 nm and 520 nm, respectively. Slit widths were 3 nm for diUb-binding assays (1 mM fluorophore) or 6 nm for tetraUb-binding assays (0.1 mM fluorophore). Anisotropies were calculated by the instrument software, and the overall intensity of the fluorescence signal was monitored at each point with the polarizers oriented at the magic angle. All measurements were made in fluorescence buffer (25 mM Na phosphate [pH 7.4], 150 mM NaCl, 5 mM β-mercaptoethanol, 1 mM EDTA, and either 0.005% surfactant P20 (BIACORE) or 0.05% Brij35). Concentrations of the fluorescent proteins were calculated using the fluorescein extinction coefficient at 494 nm, 68000 M⁻¹ cm⁻¹. (poly)Ub concentrations were assessed by absorbance at 280 nm using the extinction coefficient 0.16 (mg/ml)⁻¹ cm⁻¹ (Pickart and Raasi, 2005). Fluorescence anisotropy data were fit with a single-site binding model as described (Wilkinson, 2004). The equilibrium constants for wild-type Vps27 peptide binding to K63 and K48 Ub₄ were determined by competition with the fluorescent Vps27 7aa linker peptide as described (Wilkinson, 2004).

ITC Measurements. ITC titrations were performed on a Microcal VP-ITC at 30° C. in ITC buffer (25 mM phosphate [pH 7.4], 150 mM NaCl, 10 mM β-mercaptoethanol, and 1 mM EDTA). Each titration used 29×10 μl injections. For the titration of Rap80 tUIM peptide with K63-Ub₂, the cell contained Rap80 peptide at 250 mM and the syringe contained 2.50 mM K63-Ub₂. For the titration of K48-Ub₂, the cell contained 450 mM K48-Ub₂ and the syringe contained 4.50 mM Rap80 peptide. A version of the Rap80 tUIM peptide with a C-terminal 3-amino acid extension (DWS) was used for the ITC experiments to allow accurate peptide concentration determinations from absorbance at 280 nm.

CD Measurements. CD measurements were performed on a Jasco J-810 spectropolarimeter using a 0.2 mm pathlength cuvette. Samples were first dialyzed in 10 mM Na phosphate buffer (pH 7.4) with 100 mM NaCl. Peptide concentrations were independently determined before each CD reading from absorbance measurements (fluorescein) of samples in the CD cuvette.

### Example 1

### Rap80 UIMs Bind MonoUb Weakly

To examine Ub and polyUb binding by Rap80, a His6-tagged version of the Rap80 tUIM peptide (FIG. 1A) was produced and fluorescently labeled on a single cysteine that naturally occurs in the C-terminal UIM sequence (FIG. 1B). To resolve the contributions of the individual UIM domains to binding, single-UIM peptides were created with either of the 8-residue consensus Ub-binding sites (UIM1 and UIM2) (FIG. 1A) deleted. Because these peptides are small and relatively mobile in solution, binding to Ub or polyUb was detected as an increase in the peptide fluorescence anisotropy. Control experiments showed that the fluorescent label did not affect Ub binding (Figure S4). Typical of UIM domains (Fisher et al., 2003), UIM1 and UIM2 bound monoUb very weakly (K_{d} UIM1=510 mM, K_{d} UIM2=520 mM) (FIG. 1C). The tUIM peptide bound monoUb with the same low affinity (K_{d} tUIM=520 mM). These results indicate that both UIM domains can interact with Ub and that binding of monoUb by the tUIMs is not cooperative.

### Example 2

### Rap80 Binds Avidly Across a Single K63-Linked diUb

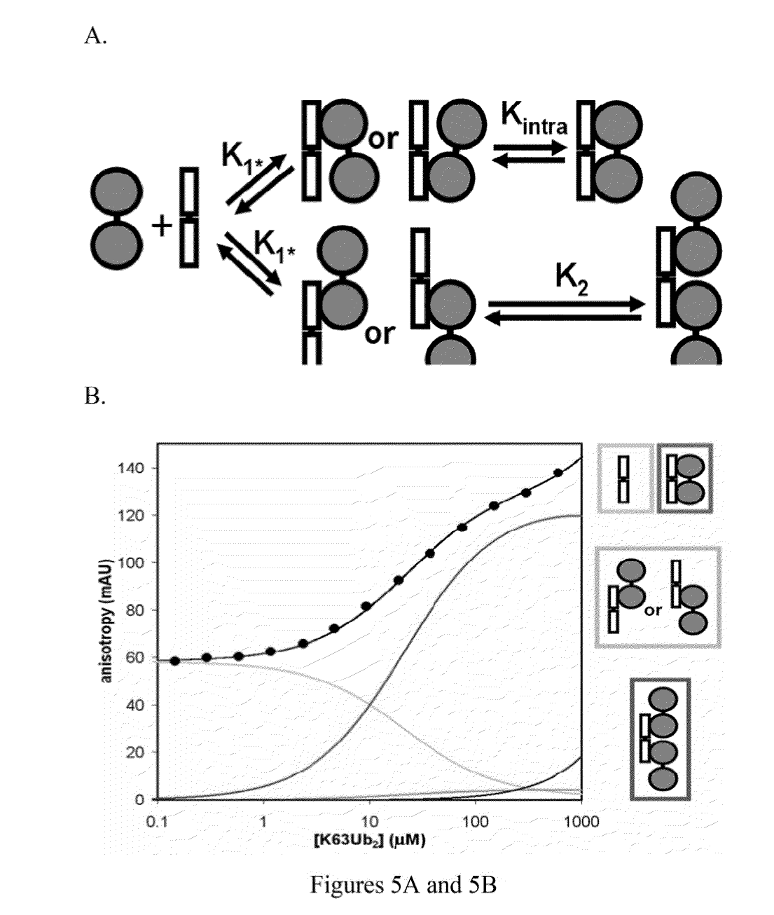

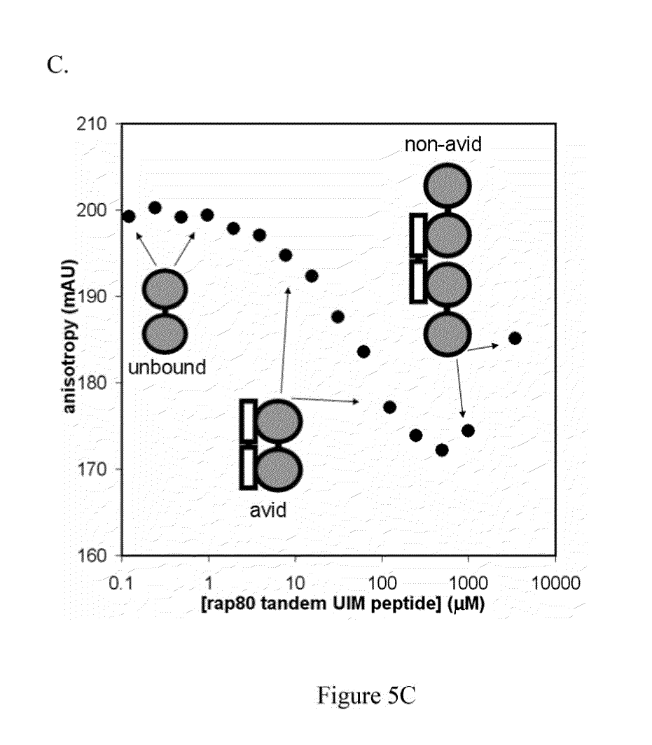

Binding of the Rap80 UIM peptides to K63-linked diUb (K63-Ub₂) was measured next. The single UIM peptides bound K63-Ub₂ with roughly the same affinity as for monoUb (K_{d} UIM1=230 mM and K_{d} UIM2=470 mM for K63-Ub₂) (FIG. 1D). In contrast, the affinity of the tUIM peptide was much tighter (K_{d} tUIM=22±1 mM for K63-Ub₂). Binding data that included higher ligand (K63-Ub₂) concentrations fit a complex model of divalent-divalent interactions well, with no change in the K_{d} value of interest (see FIG. 5).

To verify the binding constant measured by fluorescence anisotropy, isothermal titration calorimetry (ITC) of the Rap80 tUIM peptide with K63-Ub₂ was performed. The ITC data neared complete saturation and were fit by a single-site model (K_{d}^{ITC}=17.6±0.7 mM, n=0.97 sites) (FIG. 1F), in excellent agreement with the anisotropy binding constant and supporting the present interpretation of those measurements. These data demonstrate a relatively high-affinity interaction between the Rap80 tUIMs and K63-Ub₂ that depends on both UIM domains, indicating that the interaction is avid, with simultaneous binding to both Ub units in the K63-Ub₂. As a result of this avidity, Rap80 tUIMs show a 24-fold preference for K63-Ub₂ over monoUb.

### Example 3

### Rap80 Cannot Bind Avidly Across a Single K48-Linked diUb

The affinities of the Rap80 UIM peptides for K48-linked diUb (K48-Ub₂) were compared next. UIM1 and UIM2 bound K48-Ub₂ with roughly the same affinity as the tUIM peptide (K_{d}^{UIM1}=280 μM, K_{d}^{UIM2}=200 μM, K_{d}^{tUIM}=157±8 μM) (FIG. 1E). This indicates that the interaction between the tUIM peptide and K48-Ub₂ employs only one UIM domain and, therefore, is not avid.

To confirm this result, an ITC titration of K48-Ub₂ with the tUIM peptide was performed (FIG. 1G). The ITC data reveal two distinct binding sites on K48-Ub₂; both a negative and positive enthalpy interaction are evident. These data cannot be fit by a single-site model as used for the avid K63-Ub₂ interaction. Rather, the ITC data are fit well by a sequential binding model, with the first-site affinity in good agreement with the anisotropy value (K_{d}^{1,ITC}=171±40 μM) and the second-site affinity in good agreement with the interaction of a single UIM with monoUb (K_{d}^{2,ITC}=470±140 μM). It is likely that the weaker interaction was not observed in the fluorescence anisotropy titration because the ligand concentrations were lower than those used in the ITC measurement. Overall, these data indicate that the Rap80 tUIMs bind avidly to K63-Ub₂, but not across a single K48 linkage. This linkage-specific avidity underlies a 7-fold preference for K63-Ub₂ over K48-Ub₂.

### Example 4

### Closely Linked Domains Promote Affinity and Selectivity in Avid polyUb Interactions

To test the possibility that longer K48-linked chains could allow nonadjacent Ub units to interact simultaneously with a single set of tUIMs, the affinity of the tUIM peptide for K48-linked tetraUb (K48-Ub₄) was measured. The tUIM peptide bound K48-Ub₄ more than 11 times tighter than K48-Ub₂ (K_{d}^{K48-Ub4}=14 μM), an increase in affinity indicative of avid binding to Ub₄. In spite of avid interactions with longer K48 chains, the tUIM peptide still bound K63-Ub₄ with a 4-fold preference over K48-Ub₄ (K_{d}^{K63-Ub4}=3.6 μM). The decrease in K_{d} for K63-Ub₄ over K63-Ub₂ can be understood because Ub₄ contains more binding units in molar terms and because the longer chain presents a linear array of binding sites that may favor rebinding to neighboring sites. These measurements indicate that avid binding to nonadjacent Ub units is less efficient than avid binding across a single isopeptide linkage. By the same reasoning, longer linkers between UIM domains should lower affinity and thus lower linkage selectivity. To test this, the 7-amino acid sequence between the Rap80 UIMs was extended to 14 residues (REVNSQEREVNSQE). This peptide bound K63-Ub₂ substantially more weakly and with very little selectivity (Table 2).

Next, the yeast tUIM protein Vps27, which has an unstructured 25-residue linker (Swanson et al., 2003) was examined. As expected, the Vps27 tUIMs bound K63-Ub₄ and K48-Ub₄ indistinguishably. In contrast, the Vps27 UIMs connected with a 7-residue linker bound K63-linked chains with high affinity and specificity (Table 2). These results demonstrate that, for both polyUb chains and linked UBDs, binding units tightly coupled in space provide the maximum opportunity for both high-affinity and highly linkage-selective interactions.

TABLE 2
Equilibrium Dissociation Constants for Binding of tUIM Peptides to polyUb

| Tandem | | | K_{d} for | K_{d} for | K_{d} for | |
|---|---|---|---|---|---|---|
| UIM | tUIMs | Linker | K63-Ub2 | K48-Ub2 | K63-Ub4 | K_{d} for K48- |
| Peptide | Source | Sequence | (μM) | (μM) | (μM) | Ub₄ (μM) |
| Rap80 (WT) | Rap80 | REVNSQE | 21.6 ± 0.8 | 157 ± 8 | 3.6 | 14 |
| Rap80 2 x | Rap80 | 14-aa linker | 150 | 170 | — | — |
| linker | | | | | | |
| Vps27 (WT) | Vps27 | 25-aa linker | 67 | 211 | 29 | 31 |
| Vps27 7aa | Vps27 | RAEEKVK | 2.2 | 98 | 0.62 | 6.5 |
| linker | | | | | | |
| Rap80 2A | Rap80 | AA | 110 | 180 | 19 | 4.7 |
| linker | | | | | | |
| Rap80 7A | Rap80 | AAAAAAA | 3.6 | 160 | — | — |
| linker | | | | | | |
| Rap80-E | Rap80 | REVNSQ | 77 | — | — | — |
| Rap80 6aa | Rap80 | REVAAE | 87 | 160 | — | — |
| linker | | | | | | |
| Rap80 no | Rap80 | None | 47 | 130 | — | — |
| linker | | | | | | |
| Rap80 | Rap80 | REVNEQE | 16 | 170 ± 5 | — | — |
| S101E | | | | | | |
| Rap80 | Rap80 | REVNAQE | 11 | 152 ± 6 | — | — |
| S101A | | | | | | |
| Rap80 | Rap80 | DLKAAIQ | 10 | 129 ± 5 | — | — |
| DLKAAIQ | | | | | | |
| Rap80 | Rap80 | ARLEEKV | 17 | 230 | — | — |
| ARLEEKV | | | | | | |
| Rap80 | Rap80 | DM | 115 | 143 | 20 | 3.4 |
| ataxin | | | | | | |
| linker | | | | | | |
| ataxin- | ataxin3 | DM | 91 | 100 | 15 | 3.3 |
| 3 (WT) | | | | | | |
| ataxin- | ataxin3 | REVNSQE | 60 | 160 | 11 | 7.8 |
| 3 Rap80 | | | | | | |
| linker | | | | | | |
| ataxin-3 7A | ataxin3 | AAAAAAA | 17 | 140 | 3.8 | 9.3 |
| linker | | | | | | |

Replicate measurements are presented as mean ± SD. The errors associated with the fits of single titrations were typically 5%. (—) Not determined.

### Example 5

### Homology Models of Rap80 Bound to diUb Reveal the Structural Basis of Avid K63-polyUb Recognition

Multiple secondary structure prediction programs indicate that the Rap80 UIM domains are strongly a-helical, whereas the linker sequence is weakly a-helical. Circular dichroism (CD) measurements support this (FIGS. 9 and 10). FIG. 2B is a structural model based on these predictions. It was observed that, if the linking sequence acquired helical structure when bound to K63-Ub₂, the tUIM peptide would comprise one continuous a helix with an integer number of helical turns between the UIM domains. This would orient the Ub interaction surfaces of both UIMs toward the same face of the peptide in a configuration that is ideal for simultaneous interactions with both hydrophobic patches of the linear (Varadan et al., 2004) K63-Ub₂ molecule.

Next, the five structures of UIM domains bound to monoUb were examined (Hirano et al., 2006; Swanson et al., 2003; Wang et al., 2005). UIMs in all of these structures shared a common orientation on the ubiquitin surface, along the axis created by the C terminus and K63 (FIG. 2A). This would allow two UIMs on a single helix to simultaneously bind two K63-linked ubiquitins. To model this Rap80 K63Ub₂ interaction, the coordinates from the NMR structure of Vps27 UIM1 bound to monoUb (Swanson et al., 2003) were used to place each of the Rap80 UIMs on a Ub and then connected the UIMs with an α-helical segment of appropriate length. Remarkably, the resulting configuration of the bound ubiquitins easily allows a K63 linkage and is a good match for the known K63-Ub₂ structure (FIG. 7). FIG. 2D presents this model of Rap80 bound to K63-Ub₂. Consistent with the binding data, simultaneous UIM interactions could not be modeled to an open form of K48-Ub₂, even with an unstructured UIM linker (FIG. 8).

To test directly the structural transition predicted for the Rap80 linker, a version of the Rap80 tUIM peptide without additional vector-derived residues was produced and the CD spectra of the peptide in the unbound and the K63-Ub₂-bound state was measured. FIG. 2D shows the sum of the unbound Rap80 and unbound K63-Ub₂ spectra compared to the spectrum of Rap80 and K63-Ub₂ mixed at nearly saturating concentrations. Because large shifts in ubiquitin secondary structure are typically not seen upon binding, the difference in the two spectra at 222 nm indicates that Rap80 acquires significant helical structure when bound to K63-Ub₂. Whole-spectrum interpretations of these data indicate that the unbound form of Rap80 is 30%-35% α-helical, and the K63-Ub₂-bound form is nearly 100% α-helical. Simpler estimates of helical content predict less helicity (FIG. 9). These data support a model for the Rap80 K63-Ub₂ interaction in which the 7-residue linker becomes helical in the bound state and precisely positions the UIM domains for avid binding across a single K63 linkage.

In the present model of the Rap80 K63-Ub₂ interaction, direct sidechain contacts between the Rap80 linker and ubiquitin are not predicted to be important. Seven different 7-residue tUIM linkers were tested (FIG. 2E) and it was found that they all supported high-affinity, K63-specific polyUb interactions, even though a wide variety of side chains were represented at each position within the linker. This excludes a major role for direct linker side chain ubiquitin contacts in defining affinity and linkage selectivity. The common property of all of these linkers is some degree of predicted helical content, supporting the present model of the Rap80 K63-Ub₂ interaction.

### Example 6

### Factors Governing polyUb Linkage Specificity and Affinity for tUIMs

It was expected that tUIM-binding properties would be affected by both the length and structure of the linking sequence and by the intrinsic affinities of the individual UIM domains for ubiquitin. A set of peptides was constructed in which the Rap80 UIMs were linked by 1 to 9 alanine residues in order to examine linker length systematically in a way that was roughly independent from the linker structure and UIM affinity. K63-Ub₂-binding data for the alanine linker set are presented in FIG. 3A. The plot of K_{d} versus linker length is well fit by a sine wave that repeats every 3.5 residues, or approximately one a-helical turn (FIG. 3B). This is consistent with the model described herein in which a single, continuous helix of tUIMs only engages the K63-Ub₂ efficiently when the UIM-binding sites are separated by a near-integer number of helical turns and thus face the same side of the helix. The optimum linker length for K63-polyUb binding, 7 residues, also most satisfies this rule (i.e., 6.9 turns between binding sites), whereas the weakest binders (2 A and 9 A are >25-fold weaker than 7 A) have linkers that keep the UIM-binding sites almost totally out of helical phase with each other (5.6 turns and 7.5 turns between binding sites, respectively). This strong correlation demonstrates that α-helical linkers can dramatically modulate the affinity for K63-polyUb by controlling the orientation of the UIM domains with respect to each other, thus reducing or eliminating avidity.

The same pattern of affinities was observed when shorter linkers derived from the Rap80 sequence were tested (compare REVNSQ and REVAAQ to wild-type in Table 2), indicating further support for the present model of K63-Ub₂-bound Rap80. Even removing the linking sequence entirely to position the binding sites in phase reduced K63-Ub₂ affinity only 2-fold compared to wildtype, whereas the 2-residue linker “DM” binds K63-Ub₂ with the weakest affinity of all (Table 2). This may indicate some tolerance for variation in the lateral distance between UIMs but strict requirements for domain orientations. It is noted that affinity for K48-Ub₂ was largely independent of the linker length or composition in this data set and for the alanine linkers tested, consistent with the finding that K48-Ub₂ recognition is not avid (Table 2).

Intrinsic helical propensity in the linking sequence should correlate with tighter avid binding because more ordered linkers more effectively preorganize the second receptor site for binding after the first site is bound. In a striking example of this, Rap80 with the strongly helical 7-alanine linker binds K63-Ub₂ 6-fold more tightly than Rap80 with the weakly helical wild-type linker (Table 2; note no change in K48-Ub₂ affinity). CD measurements confirm the difference in helicity; in FIG. 3C, note the marked difference in the extent of the trough at 222 nm, which is one measure of helical content.

When the UIMs from ataxin-3 were spliced together with the Rap80 linker, the result was a much less helical peptide that was only weakly selective for K63-Ub₂ and showed no K63 linkage preference for longer chains. When the same UIMs were joined by the more helical 7-alanine linker, helicity and K63 selectivity were improved (FIG. 3C and Table 2). This suggests that there are minimal structural requirements for a linker in the context of its UIMs to achieve linkage specificity through avidity. This also supports the model described herein in which the Rap80 linker defines selectivity through domain positioning and not through specific contacts with Ub.

Using secondary structure prediction tools, several other linker variants were found that are predicted to have more helical content than the wild-type Rap80 linker (FIG. 10). Indeed, all of the linker variants bound K63-Ub₂ more tightly than wild-type Rap80, and the trends in affinity and selectivity closely matched the trend in predicted linker helical content (see Table 2 entries for S101A, S101E, DLKAAIQ, and ARLEEKV linkers). CD measurements confirmed the underlying difference in helical propensity for one set of these peptides, though the changes were subtle (FIG. 10). These results support the present structural model of Rap80 bound to K63-Ub₂ and indicate that linker sequences that reduce the flexibility between UIMs promote high-affinity and linkage-selective interactions.

### Example 7

### The 2-Amino Acid Linker of Ataxin-3 tUIMs Defines K48 Selectivity

Because the 7-residue linker alone accounts for most of the linkage preference of Rap80, it was questioned whether different length linkers in other tUIM proteins could define different polyUb specificities by linkage-specific avidity. At least 12 human proteins contain closely spaced tUIMs (FIG. 3D). Because ataxin-3 UIMs have a conserved 2-residue linker (FIG. 11) and because previous studies indicated that ataxin-3 binds with high affinity to K48-polyUb (Chai et al., 2004), whether the 2-residue linker conferred K48 selectivity was tested. Indeed, ataxin-3 tUIMs bound K48-Ub₄ with a 5-fold preference over K63-Ub₄ (Table 2). Remarkably, the ataxin-3 linker alone was sufficient to transfer a 6-fold K48 preference to the Rap80 UIM domains (Table 2). The effect was apparently not due to linker specific side-chain contacts with ubiquitin, as a 2-alanine linker between the Rap80 UIMs conferred K48-Ub₄ specificity nearly as well (Table 2).

With 7-residue linkers, differences in avidity and, thus, linkage specificity were apparent for differently linked diubiquitins. In contrast, for 2-residue linkers, interactions with Ub₂ were apparently not avid for either linkage and were of low affinity and negligible selectivity (Table 2). Instead, high-affinity, avid binding was seen only for longer chains, where K48 linkage specificity was evident. This indicates that receptors can distinguish polyUb linkages by avidity when the unit of avid recognition is longer polyUb chains. It is noted that the 2-alanine linker form of the Rap80 tUIM bound K63-Ub₂ the most weakly of all the tUIMs in the alanine linker series. Further binding and structural studies are required to determine whether 2-residue linkers position UIM domains for truly optimum K48-polyUb binding or whether this arrangement simply excludes K63-polyUb most efficiently.

### II. Avid Interactions Underlie The K63-Linked Polyubiquitin Binding Specificities Observed for UBA Domains

Ubiquitin (Ub) receptor proteins as a group must contain a diverse set of binding specificities to distinguish the many forms of polyUb signals. Previous studies suggested that the large class of ubiquitin associated (UBA) domains contains members with intrinsic specificity for lysine 63-linked polyUb (K63-polyUb) or K48-polyUb, thus explaining how UBA-containing proteins can mediate diverse signaling events. As described herein, the previously observed K63-polyUb selectivity in UBA domains is the result of an artifact in which the dimeric fusion partner, glutathione-S-transferase (GST), positions two UBAs for higher affinity, avid interactions with K63-polyUb, but not K48-polyUb. Freed from GST, these UBAs are either non-selective or prefer K48-polyUb. Accordingly, NMR experiments reveal no K63-polyUb specific binding epitopes for these UBAs. Previous conclusions based on GST-UBAs are re-examined and an alternative model is presented for how UBAs achieve a diverse range of linkage-specificities.

### Materials and Methods

Plasmids and Proteins. All GST-UBA proteins used were previously reported (Rassi et al., 2005). The sequences of all UBA proteins used in this study are shown in Table 3, along with cloning details. For NMR studies, the Ede1 UBA domain was cleaved from the GST-fusion protein using thrombin protease according to the manufacturer's instructions. Some NMR experiments used a His-tagged version of Ede1 UBA, with the affinity tag intact (‘His10-Ede1 UBA’). The behavior of this protein was essentially identical to the domain cleaved from GST with respect to Ub binding and NMR spectral properties. For NMR studies of hHR23A UBA1, the UBA domain was cleaved from the GST fusion using thrombin. All proteins were expressed in E. coli. GST-fusions were purified on glutathione agarose (Sigma) according to the manufacturer's instructions. His-tagged proteins were purified on Ni-NTA agarose (Qiagen) according to the manufacturer's instructions, followed by anion-exchange or gel filtration chromatography when needed. UBA domains for expressed protein ligation were purified on chitin beads (New England Biolabs), with additional purification by anion-exchange or gel filtration chromatography when needed after ligation. MonoUb and polyUb chains were prepared as described in Pickart et al., 2004. For binding studies that used K48 or K63 chains, the proximal Ub blocking residue D77 was left in place, and the distal Ub contained a single lysine-to-arginine substitution at position 48 or 63, respectively. Radiolabeled polyUb chains for pull-down assays were produced as described (Pickart et al., 2004). NMR samples of Ede1 UBA, hHR23A UBA-1, monoUb and Ub₂ (protein concentrations 0.35-0.8 mM) were prepared in the appropriate buffers containing 20 mM sodium phosphate at pH 6.8, 7% (v/v) D₂O, and 0.02% (w/v) NaN₃. Except for spin-labeling studies, all Ede1 UBA samples contained 5 mM β-mercaptoethanol.

TABLE 3
Sequence and Cloning Details for Proteins

| Protein | | Cloning | | |
|---|---|---|---|---|
| Name | Vector | Sites | Sequence | Use |
| GST-Edel | pGEX4 | BamHI | (GST)-GSATTPKSLAVEELSGMGFTEEEAHNA | Pull- |
| UBA | T2 (GE) | SalI | LEKCNWDLEAAT NFLLDSA | downs, SPR, |
| | | | | NMR (GST cleaved) |
| GST-hHR23 | pGEX4 | BamHI | (GST)-GSTGSEYETMLTEIMSMGYERERVVAA | SPR, NMR |
| A | T2 (GE) | SalI | LRASYNNPHRAVEYLLTGIPGSPEPEHGLGRLER | (GST cleaved) |
| UBA1 | | | PHRD | |
| GST-Ubcl | pGEX4 | BamHI | (GST)-GSAGIDHDLIDEFESQGFEKDKIVEVRR | SPR |
| UBA | T2 (GE) | SalI | LGVKSLDPNDNNTANRIIEELLKS | |
| GST-DRM2 | pGEX4 | BamHI | (GST)-GSEAGSSKSKAIDHFLAMGFDEEKVVKA | SPR |
| UBA1 | T2 (GE) | SalI | IQEHGEDNMEAIANALLSCPEAK | |
| His10-Edel | PET 16b | NdeI | MGHHHHHHHHHHSSGHIEGRHMATTPKSLAV | NMR |
| UBA | (Novagen | BamHI | EELSGMGFTEEEAHNALEKCNWDLEAATNFLL | |
| | | | DSA | |
| Edel | pTYB2 | NdeI | MATTPKSLAVEELSGMGFTEEEAHNALEKCNW | Fluor. |
| UBA | (NEB) | SmaI | DLEAATNFLLDSAG-(intein) | anisotropy |
| hHR23 A | pTYB2 | NdeI | MTGSEYETMLTEIMSMGYERERVVAALRASYN | Fluor. |
| UBA | (NEB) | SmaI | NPHRAVEYLLTGG-(intein) | anisotropy |
| Dsk2 | pTYB2 | NdeI | MPPEERYEHQLRQLNDMGFFDFDRNVAALRRS | Fluor. |
| UBA | (NEB) | SmaI | GGSVQGALDSLLNGSG-(intein) | anisotropy |
| Ddi1 | pTYB2 | NdeI | MTFPEQTIKQLMDLGFPRDAVVKALKQTNGNA | Fluor. |
| UBA | (NEB) | SmaI | EFAASLLFQSG-(intein) | anisotropy |
| Ubcl | pET16b* | NdeI | MPHHHHHHHHHHSSGHIEGRHMAGIDHDLIDE | Fluor. |
| UBA | (Novagen) | BamHI | FESQGFEKDKIVEVLRRLGVKSLDPNDNNTANR | anisotropy |
| | | | IIEELLKSC | |

*A version of pET 16b was used in which the second amino acid in the vector-derived N-terminal tag was mutated from G to P.

Fluorescent Labeling. Ede1 UBA, hHR23A UBA1, Dsk2 UBA and Ddi1 UBA were produced as intein fusions and fluorescently labeled using expressed protein ligation as described in Scheibner et al., 2003. The dipeptide used for Ede1 labeling was Cys-Lys, with NHS-rhodamine (Pierce) coupled to the lysine amino group, and was a kind gift from Philip Cole (Johns Hopkins School of Medicine). MALDI mass spectroscopy confirmed the addition of the fluorescent dipeptide to the Ede1 UBA domain and that there was complete cleavage of the initiating methionine residue. hHR23A UBA1, Dsk2, and Ddi1 UBAs were labeled in a similar way, except that the dipeptide contained a fluorescein moiety instead of rhodamine. The fluorescein-labeled dipeptide was made as described in Scheibner et al., 2003. Ubc1 UBA from yeast was expressed with an N-terminal His-tag and an additional C-terminal Cys. Fluorescent labeling was achieved with Alexa-fluor 546 maleimide (Invitrogen) following the manufacturer's instructions.

Pull-Down Assays. GST-UBA protein was added to 15 μl of glutathione-agarose and the beads were washed with binding buffer [25 mM phosphate pH 7.4, 150 mM NaCl, 10 mM β-mercaptoethanol, 1 mM EDTA, 0.05% (v/v) Brij35]. ¹²⁵I-labeled K63 or K48-Ub₄ (1 μM) was then added in 100 μl of binding buffer plus 1 mg ml⁻¹ BSA, and the beads agitated gently for 20 min. at room temperature. The specific radioactivities of ¹²⁵I-labeled K63- and K48-Ub₄ were normalized beforehand with unlabeled chains. The beads were then washed quickly 2 or 3 times with binding buffer. The bound chains were eluted with SDS-PAGE sample buffer and resolved by SDSPAGE. The Ub₄ bands were excised from the gel and quantified with a gamma-counter. “Bound counts” in FIG. 12d and FIG. 15d is the radioactivity (cpm) for a particular band minus the counts in the corresponding gel slice in the negative-control lane.

Surface Plasmon Resonance. SPR analyses was performed on a Biacore 3000 instrument at 25° C. in HBS-EP buffer (Biacore). Anti-GST antibody (Biacore) was immobilized by amine coupling on a Biacore CM5 chip. GST-UBA proteins were captured on a measurement surface at a density of 150-400 RU; an antibody-coupled surface served as the reference. Ubₙ chains were applied to the chip with a 5 μl/min flow rate and recorded the response; 50 mM glycine pH 1.8 or 15 mM NaOH was used to remove GST-UBA proteins and renew the surface. The data was fit with a single-site binding model as described in Wilkinson, 2004.

Fluorescence Anisotropy Binding Assays. Fluorescence anisotropy measurements were performed as described (Sims and Cohen, 2009) using excitation and emission maxima of 492 nm/520 nm (fluorescein), 556 nm/569 nm (Alexa Fluor 546), or 555 nm/578 nm (rhodamine), in binding buffer at 25° C. The concentrations of the fluorescent proteins were calculated using published extinction coefficients (Invitrogen-Molecular Probes). polyUb concentrations were assessed from absorbance at 280 nm using the Ub extinction coefficient of 0.16 (mg per ml)⁻¹ cm^{'1.49}. The data was fit with a single-site binding model (Wilkinson, 2004).

NMR Methods. All NMR studies were performed on a cryoprobe-equipped Bruker 600 MHz spectrometer at 23° C. NMR samples of Ede1 UBA, hHR23A UBA1, monoUb and Ub₂ (protein concentrations 0.35-0.8 mM) were prepared in the appropriate buffers containing 20 mM sodium phosphate at pH 6.8, 7% (v/v) D₂O, and 0.02% (w/v) NaN₃. In addition, all Ede1 UBA samples contained 5 mM β-mercaptoethanol. ¹⁵N-labeled Ub₂ chains were synthesized segmentally (Varadan et al., 2008; Varadan et al., 2002). NMR signal assignments for monoUb and Ub₂ at pH 6.8 were from previous studies (Varadan et al., 2008; Varadan et al., 2002). NMR signal assignments for the Ede1 UBA and hHR23A UBA1 domains were from the literature (Swanson et al, 2006; Mueller et al, 2002). NMR data were processed using XWINNMR and analyzed using the program CARA and in-house software.

Binding-interface mapping was achieved in a series of NMR titration experiments in which 2D ¹H-¹⁵N HSQC or SOFAST spectra of a ¹⁵N-labeled species of interest (e.g., Ede1 UBA) were recorded as a function of the increasing amount of unlabeled binding partner (e.g., Ub₂). To map the binding surface on a specific Ub unit in Ub₂, a similar assay was performed in which unlabeled UBA was added to segmentally ¹⁵N-labeled Ub₂. Binding was monitored through accompanying changes in the peak positions in 2D ¹H-¹⁵N HSQC spectra and quantified using combined amide chemical shift perturbation (CSP) calculated as Δδ=[(ΔδH)²+(ΔδN/5)²]^{1/2}, where ΔδH and ΔδH are the observed chemical shift changes for ¹H and ¹⁵N, respectively. To monitor site-specific changes in NMR signal intensities due to line broadening (as a result of intermediate or slow exchange), the NMR spectra obtained in the course of titration were uniformly scaled to compensate for the higher molecular weight of the complex. The signal attenuation was then calculated for each residue as the ratio of peak intensities in the corresponding spectra of the free and bound protein.

To determine the UBA-binding surface on the distal or proximal Ub in Ub₂, 0.35 and 0.6 mM ¹⁵N-labeled K48-linked Ub₂ samples (Ub₂-D or Ub₂-P, respectively) were titrated with increasing amounts of unlabeled Ede1 UBA (from a stock solution). Titration for the proximal and distal Ub continued up to an Ede1 UBA:Ub₂ molar ratio of 4.0. Similar studies were performed with ¹⁵N-labeled K63-linked Ub₂-D (0.5 mM) and Ub₂-P (0.75 mM) samples up to a Ede1 UBA:Ub₂ molar ratio of 2.9 and 4.0, respectively. Two sets of the reverse titration experiments were performed. The first set was at lower concentrations, with 0.2 mM and 0.4 mM ¹⁵N-labeled Ede1 UBA samples titrated with increasing amounts of unlabeled K63- and K48-linked Ub₂ to give [Ub₂]/[Ede1 UBA]=2.9 and 5.5, respectively. The second set was at higher concentrations, with 0.6 and 0.61 mM ¹⁵N-labeled UBA titrated with K63- and K48-linked Ub₂s, respectively, up to [Ub₂]/[Ede1 UBA]=3.5. As control experiments, increasing amounts of unlabeled Ede1 UBA were titrated into a 0.8 mM ¹⁵N-labeled monoUb sample up to a Ede1 UBA:Ub₂ molar ratio of 3.0, and unlabeled monoUb was titrated into a 0.45 mM ¹⁵N-labeled Ede1 UBA sample up to a Ub:UBA molar ratio of 2.8. Similar procedures were used to map the surface on hHR23A UBA1 involved in binding to monoUb or K48-linked Ub₂.

### Example 8

### GST-Ede1 UBA Preferentially Binds K63-Linked polyUb

To identify a model K63-selective UBA domain for structural studies, 4 of the 7 UBA domains classified as K63-selective by Raasi et al. were examined. The GST-fused minimal UBA domain constructs from their original study were used to assay binding to K63- and K48-Ub₂ using surface plasmon resonance (SPR). The equilibrium dissociation constants (K_{d}s) determined are shown in Table 4, and representative binding curves for GST-Ede1 UBA (yeast) binding to K63-Ub₂ and K48-Ub₂ are shown in FIG. 12A. All four GST-UBA fusion proteins bound K63-Ub₂ more tightly than K48-Ub₂. Binding to Ub₄ for the two UBAs with the highest affinity, GST-Ede1 UBA and GST-hHR23A UBA1 (human) was examined next. GST-Ede1 UBA was more linkage selective, and so became a model K63-selective UBA domain (4.2-fold selective for K63-Ub₄; Table 4 and FIG. 12B). The magnitude of this selectivity is comparable to other linkage-selective UBA domains (e.g., about 5-fold K48 preference by hHR23A UBA₂) (Rassi et al., 2005; Rassi et al, 2004).

The differences between the measured and theoretical SPR values (residuals) for the Ub₄ ligands are plotted in FIG. 12B (lower panel). For K48-Ub₄, the small and random residuals indicate that the data are well described by the 1:1 interaction model used in the fit. For K63-Ub₄ binding, however, large and non-random residuals are evident, potentially indicating more complex binding modes. SPR data for GST-Ede1 UBA vs. the Ub₂ ligands (FIG. 12A) and GSThHR23A UBA1 vs. Ub₂ and Ub₄ exhibit similar patterns of residuals, although the magnitude of the systematic deviations is smaller. It is noted that this pattern of non-random error in the 1:1 fit is frequently a feature of SPR data for UBA•polyUb binding, particularly when the UBA domain is the immobilized binding partner (unpublished results; Trempe et al., 2005; Raasi et al., 2004).

Because GST pull-down assays are the most common technique used to evaluate polyUb linkage specificity, a quantitative version of a pull-down assay using GSTEde1 UBA was also performed to capture radiolabeled Ub₄ chains for comparison to the SPR data. By this method, the preference of GST-Ede1 for K63-Ub₄ over K48-Ub₄ appeared to be even larger (12-fold K63 selective by pull-down, FIG. 12C, D).

TABLE 4
Dissociation Equilibrium Constants (K_{d}) for GST-UBA Domains
Interacting With Polyubiquitins

| GST-UBA | K63-Ub₂ | K48-Ub₂ | K63-Ub₄ | K48-Ub₄ |
|---|---|---|---|---|
| Ede1 | 48 ± 9 | 110 ± 16 | 6.0 ± 1.9 | 25 |
| hHR23A UBA1 | 26 | 36 | 4.7 | 13 |
| Ubc1 | 130 | 460 | ND | ND |
| DRM2 UBA1 | 89 | 140 | ND | ND |

K_{d} values (μM) were determined by SPR. Measurements with 3 or 4 replicates are presented as (K_{d} ± standard deviation).
ND, not determined.

### Example 9

### The Mode of Ede1 UBA Binding is not Linkage-Specific

NMR backbone amide (¹H, ¹⁵N) chemical shift perturbation (CSP) studies were next performed to gain insight into the molecular basis of K63-selective binding. ¹⁵N-labeled Ede1 was titrated with monoubiquitin, K63-Ub₂, or K48-Ub₂ to determine the residues responsible for each of these interactions (FIG. 13A-C). The observed CSPs for monoUb•Ede1 UBA agreed well with published data for this interaction (Swanson et al., 2006). However, the Ede1 CSP maps with all three ligands were nearly identical, with the primary interaction surface composed of residues on helix 1 and helix 3 of the UBA domain. Though the chemical shifts for residues E1364 and K1365 are slightly more perturbed in the complex with K63-Ub₂ than with K48-Ub₂ or monoUb (FIG. 13B), binding studies with E1364A and K1365A UBA domains indicated that these residues are not important determinants of binding or selectivity (FIG. 18). This result is in contrast to the interaction of hHR23A UBA₂ with its preferred binding partner, K48-Ub₂13. In this model of selective polyUb binding, an additional, distinct interface on UBA₂ was evident from the titration with K48-Ub₂ but not with K63-Ub₂ or monoUb.

The inverse experiments were performed next using versions of Ub₂ with either the proximal (free C-terminus) or distal (free lysine 48 or 63) Ub selectively labeled with ¹⁵N and titrated with unlabeled Ede1 UBA. (¹H, ¹⁵N) monoUb CSPs were collected for comparison to the polyUb data (FIG. 13D-G). These data reveal that, contrary to the expectations for a K63-selective UBA, both the proximal and distal Ub CSPs from the K63-Ub₂•Ede1 UBA interaction are essentially the same as those observed from monoUb binding. For Ede1•K48-Ub₂, the distal Ub CSPs were like those for both Ubs in K63-Ub₂ and for monoUb. Significant differences were seen only in resonances from the proximal Ub of the K48-Ub₂ titration, where the overall magnitude of CSPs was lower than in the other titrations (FIG. 19). This phenomenon has been observed for other K48-Ub₂ interactions and may relate to the opening closing dynamics of the K48-Ub₂ chain (Hairinia et al., 2007; Varadan et al., 2005). These results indicate that Ede1 UBA interacts with polyUb in a non-linkage-selective manner, in contrast to the SPR and pull-down results with GST-Ede1 UBA.

### Example 10

### Free Ede1 UBA Domain is not Linkage-Selective

To examine polyUb specificity of the free Ede1 UBA domain, a fluorescent version of the minimal domain free of affinity tags was produced by expressed protein ligation (Scheibner et al., 2003) (Ede1_rhodamine), and binding to polyUb was measured by fluorescence anisotropy. In contrast to the >4-fold K63 selectivity of the GST-Ede1 UBA, Ede1_rhodamine is not linkage-selective (FIG. 14A, B and Table 5). This is in agreement with the CSP-mapping result that Ede1 UBA binds to K63-polyUb, K48-polyUb, and monoUb with virtually identical interfaces. The small preference (<2-fold) of Ede1_rhodamine for K48-polyUb has been observed for other UBDs that bind in non-linkage-specific configurations (Sims et al., 2009; Varadan et al., 2002). In K48-linked diUb, the residues on the Ub surface that are required for UBA interaction can face each other in a deep pocket (Varadan et al., 2002). For a UBA bound to a K48-linked Ub, non-specific contacts with an adjacent Ub in the chain might facilitate rebinding after dissociation, explaining the small observed preference for K48-Ub₂. Another possibility is that the compact, closed form of K48-Ub₂ presents a slightly smaller entropic barrier to binding than the relatively flexible K63-linked Ub dimer.

K48-polyUb Kds agreed closely between GST-Ede1 and Ede1_rhodamine measurements (Tables 4 and 5). The large deviations in K63-polyUb affinities between the two constructs (9-fold for Ub4) suggested that GST-fusion can artificially promote UBA•K63-polyUb interactions. It was suspected that the dimeric GST moiety of the GST-fusion could bring together two UBA domains in a configuration that promotes simultaneous or avid binding to a single K63-linked chain, but not to K48-polyUb. Because avid interactions are potentially more favorable, this could lead to the apparent linkage selectivity observed for some GST-UBAs. This mechanism, termed “linkage-specific avidity”, can determine the polyUb linkage preference for sets of ubiquitin interacting motifs (UIMs) that are held close in space by a short linking sequence (Sims and Cohen, 2009). Modeling suggests that two GST-fused UBAs could interact avidly with adjacent Ubs in a chain (FIG. 20).

For GST-UBAs, a tighter, avid binding mode would contribute to binding at ligand (Ubn) concentrations below the intrinsic UBA•Ub Kd. At ligand concentrations nearer to the intrinsic Kd, each UBA could bind a separate chain. This mixed mode of binding would explain the systematic deviations from the 1:1 model that have been observed for some GST-UBAs interacting with K63-polyUb (FIG. 12A, B), i.e., more binding at low ligand concentrations than can be accounted for by the 1:1 binding model. In support of this hypothesis, it is noted that the free UBA binding data do not exhibit these same systematic deviations (FIG. 14A, B).

TABLE 5
Dissociation Equilibrium Constants (K_{d}) for Fluorescent UBA
Domains Interacting With Polyubiquitins

| UBA | K63-Ub₂ | K48-Ub₂ | K63-Ub₄ | K48-Ub₄ |
|---|---|---|---|---|
| Ede1 | 120 | 86 | 54 | 31 |
| hHR23A UBA1 | ND | ND | 30 | 9.0 |
| Ubc1 | ND | ND | 170 | 37 |
| Dsk2 | ND | ND | 2.2 | 1.4 |
| Ddi1 | ND | ND | 92 | 50 |

K_{d} values (μM) were determined by fluorescence anisotropy.
ND, not determined.

### Example 11

### Bivalency Accounts for Linkage Selectivity in GST-Ede1 UBA

To test whether the bivalency of the GST-Ede1 UBA is responsible for its higher K63-polyUb affinity, a GST dimer that contained only one UBA fusion polypeptide was created. This was accomplished by taking advantage of the slow exchange of subunits between GST dimmers (Scheibner et al., 2003; Kaplan et al., 1997). First, a GST protein with a 6-His affinity tag was produced, but no UBA fusion (GST-His). GST-His was mixed with GST-Ede1 at a 12:1 mole ratio, the mixture was unfolded in 6 M urea, and then refolded by rapid 10-fold dilution into urea-free buffer. In a final step, dimers with a His tag were purified on Ni²⁺-NTA agarose, thus removing any reformed GST-Ede1/GSTEde1 homodimers (FIG. 15A). The mixture obtained, termed GST-fmm for “functionally monomeric mixture,” contained GST-His homodimers and GST-His/GST-Ede1 heterodimers at a ratio of about 6:1. The GST-Ede1/GST-Ede1, GST-His/GST-His, and GST-His/GST-Ede1 dimers could be separated by native PAGE because of charge differences between the two component polypeptides (FIG. 15B). The large excess of GST-His homodimers in the mixture was intended to reduce artificial multivalency between adjacent, non-dimerized UBAs in assays such as pull-downs and SPR where proteins are immobilized at high density on a solid surface.

GST-Ede1 to GST-fmm was compared by pull-down assay with radiolabeled Ub₄ chains (FIG. 15C). GST-Ede1 and GST-fmm samples were loaded onto glutathione-agarose so that equal amounts of GST-Ede1 polypeptides were on the beads. Because the resin with GST-fmm contained more total protein due to the excess GST-His, separate negative controls with GST-His alone were performed. Similar to previous pull-down assay results (FIG. 12C, D), GST-Ede1 was >11-fold selective for K63-Ub₄ over K48-Ub₄. In contrast, GST-fmm bound Ub₄ chains with no linkage preference (FIG. 15D). Note that the GST-fmm and GST-Ede1 bound nearly identical amounts of K48-Ub₄. This suggests that affinity for K48 chains is largely independent of the oligomeric state of the Ede1 UBA, and supports the idea that the linkage selectivity of dimeric GST-Ede1 arises from avid interactions that are only possible with K63-polyUb.

Using the SPR assay, GST-fmm bound K63-Ub₄ more weakly than GST-Ede1 and had reduced linkage selectivity (FIG. 15E; Kd K63 Ub₄=23 μM, Kd K48 Ub₄=44 μM). The small difference between these SPR Kds and those derived from the fluorescence assays with free UBA (Table 5) may be the result of some inevitable multivalency generated by neighboring heterodimers on the surface of the SPR chip. The systematic error associated with SPR measurements of GST-Ede1•K63-Ub₄ was reduced but not eliminated for GST-fmm (FIG. 15E). If this indicates a link between multivalent binding and deviations from the 1:1 fit, then it indeed seems that the GST-fmm mixture reduced but did not totally eliminate multivalency in the context of the SPR assay. Nonetheless, it is clear from these measurements that the additional valency in the GST-Ede1 homodimer is responsible for most of the artifactual K63-polyUb affinity.

### Example 12

### GST-Fusion Inflates K63-polyUb Binding for Other UBAs

It was suspected that this artifactual K63-selectivity may apply to GST-hHR23A-UBA1 as well. A fluorescein-labeled hHR23A-UBA1 domain was produced free of affinity tags and its interactions with Ub₄ chains were measured (FIG. 16A). In contrast to the K63-selectivity observed for GSTUBA1, the free domain actually has a small but significant preference for K48-linked polyUb (FIG. 16A, Table 5). As with Ede1 UBA, K48-polyUb affinities agree for the two constructs, but K63-polyUb affinities do not (the GST-UBA1 is >6-fold tighter for K63-Ub₄ than the free domain, Tables 4 and 5).

NMR CSPs were again mapped to investigate the molecular determinants of this preference. ¹⁵N-labeled UBA1 was titrated with unlabeled monoUb or K48-Ub₂. These experiments revealed that, as with most UBA domains, the interaction with monoUb is mediated by residues primarily on one face of the domain, comprising helix 1 and helix 3 (FIG. 16B). The interaction with K48-Ub₂, however, involves an extended surface that contains residues on the helix 2-helix 3 side of the domain in addition to the helix 1-helix 3 interface (FIG. 26C). This observation closely matches the published results for UBA₂ from the same protein, which is K48-selective (Varadan et al., 2005). For UBA₂, the helix 2-helix 3 contacts are part of a K48-specific epitope that is fully engaged only when the UBA is “sandwiched”’ between the Ubs of K48-Ub₂. The results suggest that UBA1 achieves K48-linkage preference in the same way, with an extended interface that can be completely occupied only in the complex with K48-Ub₂. Aligning the coordinates for free UBA1²⁰ to the K48-Ub2•UBA₂ complex (Varadan et al., 2005) shows how the K48-specific contacts on UBA1 could engage K48-Ub₂ along the isopeptide bond and proximal Ub (FIG. 16D). As with Ede1 UBA, the structural details of the interaction are consistent with the binding preference of the free domain, and not the GST-fused construct. Thus, it is likely that the smaller K63-selectivity we observed for GST-fused hHR23A-UBA1 (Table 4) is the result of competition between the intrinsic K48-selectivity of the domain and the avidity-driven K63-selectivity of the GST-fusion.

Another UBA from the K63-selective GST fusions, Ubc1 UBA (yeast), bound with an even larger K48-preference when expressed as a free domain (Table 5). However, it was found that GST fusion does not necessarily result in the overestimation of K63-polyUb affinity for all GST-UBAs. Both GST-Dsk2 UBA (yeast) and. GST-Ddi1 (yeast) UBA were originally shown to bind polyUb without a linkage preference (Raasi et al., 2005); these experiments indicate that the free UBAs are indeed non-selective (<2-fold difference in Kd for K63- and K48-Ub₄, Table 5). In support of these results, the human homolog of Dsk2, ubiquilin-1, was found to bind without linkage selectively to K63- and K48 polyUb in a manner similar to its interaction with monoUb (Zhang et al., 2008).

One possible difference between the UBA domains prone to the K63-GST artifact and those that are not may be the degree or nature of UBA self-association. Forced proximity from a dimerized GST (FIG. 20) could promote even weak or non-specific self-association of some UBAs, resulting in a conformation that favors avid binding to adjacent, K63-linked Ubs. Several UBA domains have been shown to self-associate (Kozlov et al., 2007; Bayrer et al., 2005); and Prag et al., 2003), and in at least one instance this UBA-UBA interaction promotes polyUb binding (Peschard et al., 2007). Some evidence that Ede1 UBA domain may self-associate at high concentrations and in the context of the GST-fusion has been collected (data not shown), though the present studies were not conclusive about the contribution this property makes to apparent linkage selectivity.

Intriguingly, Dsk2 oligomerization has been suggested to play a part in K48-polyUb selectivity (Lowe et al., 2006). Full-length Dsk2 self-associates (Sasaki et al., 2005) and, by inference, has shown an in vivo binding preference for K48-linked polyUb (Matiuhin et al., 2008). However, no linkage selectivity for the isolated domain has been observed under conditions that favor dimerization (i.e., as a GST fusion (Raasi et al., 2005)) or conditions that should prevent UBA self-association (i.e., use of low UBA concentrations in fluorescence binding assays). It is likely that the precise configuration of self-associated UBAs would influence linkage selectivity. This property of some UBAs could be either functionally relevant or an artifact of some assays. Detailed biophysical studies will be required to determine the contributions of UBA domain self-association to linkage-selective binding.

### REFERENCES

1. Aguilar R C, Watson H A, Wendland B. The yeast Epsin Ent1 is recruited to membranes through multiple independent interactions. J Biol Chem 2003; 278:10737-10743.

2. Andrade, M. A., Chacon, P., Merelo, J. J., and Moran, F. (1993). Evaluation of secondary structure of proteins from UV circular dichroism spectra using an qq unsupervised learning neural network. Protein Eng. 4, 383-390.

3. Barriere, H., Nemes, C., Lechardeur, D., Khan-Mohammad, M., Fruh, K., and Lukacs, G. L. (2006). Molecular basis of oligoubiquitin-dependent internalization of membrane proteins in Mammalian cells. Traffic 7,282-297.

4. Bayrer J R, Zhang W, Weiss M A. Dimerization of double sex is mediated by a cryptic ubiquitin associated domain fold: implications for sex-specific gene regulation. J Biol Chem 2005; 280:32989-32996.

5. Bennett, E. J., and Harper, J. W. (2008). DNA damage: ubiquitin marks the spot. Nat. Struct. Mol. Biol. 15, 20-22.

6. Bobrovnik, S. A. (2007). The influence of rigid or flexible linkage between two ligands on the effective affinity and avidity for reversible interactions with bivalent receptors. J. Mol. Recognit. 20, 253-262.

7. Broemer M, Meier P. Ubiquitin-mediated regulation of apoptosis. Trends Cell Biol. 2009

8. Bjorkoy G, Lamark T, Johansen T. p62/SQSTM1: a missing link between protein aggregates and the autophagy machinery. Autophagy 2006; 2:138-139.

9. Bjorkoy G, et al. p62/SQSTM1 forms protein aggregates degraded by autophagy and has a protective effect on huntingtin-induced cell death. J Cell Biol 2005; 171:603-614.

10. Chai, Y., Berke, S. S., Cohen, R. E., and Paulson, H. L. (2004). Poly-ubiquitin binding by the polyglutamine disease protein ataxin-3 links its normal function to protein surveillance pathways. J. Biol. Chem. 279, 3605-3611.

11. Chen Z, Pickart C M. A 25-kilodalton ubiquitin carrier protein (E2) catalyzes multi-ubiquitin chain synthesis via lysine 48 of ubiquitin. J Biol Chem 1990; 265:21835-21842.

12. Di Francesco, V., Garnier, J., and Munson, P. J. (1996). Improving protein secondary structure prediction with aligned homologous sequences. Protein Sci. 1, 106-113.

13. Fisher, R. D., Wang, B., Alam, S. L., Higginson, D. S., Robinson, H., Sundquist, W. I., and Hill, C. P. (2003). Structure and ubiquitin binding of the ubiquitin-interacting motif. J. Biol. Chem. 278, 28976-28984 effective affinity and avidity for reversible interactions with bivalent receptors. J. Mol. Recognit. 20, 253-262.

14. Fisher, R. D., Wang, B., Alam, S. L., Higginson, D. S., Robinson, H., Sundquist, W. I., and Hill, C. P. (2003). Structure and ubiquitin binding of the ubiquitin-interacting motif. J. Biol. Chem. 278, 28976-28984.

15. Friedlander R, Jarosch E, Urban J, Volkwein C, Sommer T. A regulatory link between ER-associated protein degradation and the unfolded-protein response. Nat Cell Biol 2000; 2:379-384.

16. Gagny B, et al. A novel EH domain protein of Saccharomyces cerevisiae, Ede1p, involved in endocytosis. J Cell Sci 2000; 113(Pt 18):3309-3319.

17. Greenfield, N. J. (2006). Using circular dichroism spectra to estimate protein secondary structure. Nat. Protoc. 6, 2876-2890.

18. Gyrd-Hansen M, et al. IAPs contain an evolutionarily conserved ubiquitin-binding domain that regulates NF-kappaB as well as cell survival and oncogenesis. Nat Cell Biol 2008; 10:1309-1317.

19. Haglund, K., Sigismund, S., Polo, S., Szymkiewicz, I., Di Fiore, P. P., and Dikic, I. (2003). Multiple monoubiquitination of RTKs is sufficient for their endocytosis and degradation. Nat. Cell Biol. 5, 461-466.

20. Haririnia A, D'Onofrio M, Fushman D. Mapping the interactions between Lys48 and Lys63-linked di-ubiquitins and a ubiquitin-interacting motif of S5a. J Mol Biol 2007; 368:753-766.

21. Hawryluk, M. J., Keyel, P. A., Mishra, S. K., Watkins, S. C., Heuser, J. E., and Traub, L. M. (2006). Epsin 1 is a polyubiquitin-selective clathrin-associated sorting protein. Traffic 7, 262-281.

22. Hicke, L., and Dunn, R. (2003). Regulation of membrane protein transport by ubiquitin and ubiquitin-binding proteins. Annu. Rev. Cell Dev. Biol. 19, 141-172.

23. Hicke, L., Schubert, H. L., and Hill, C. P. (2005). Ubiquitin-binding domains. Nat. Rev. Mol. Cell Biol. 6, 610-621.

24. Hirano, S., Kawasaki, M., Ura, H., Kato, R., Raiborg, C., Stenmark, H., and Wakatsuki, S. (2006). Double-sided ubiquitin binding of Hrs-UIM in endosomal protein sorting. Nat. Struct. Mol. Biol. 13, 272-277.

25. Hofmann, K., and Falquet, L. (2001). A ubiquitin-interacting motif conserved in components of the proteasomal and lysosomal protein degradation systems. Trends Biochem. Sci. 26, 347-350.

26. Huen, M. S., Grant, R., Manke, I., Minn, K., Yu, X., Yaffe, M. B., and Chen, J. (2007). RNF8 transduces the DNA-damage signal via histone ubiquitylation and checkpoint protein assembly. Cell 131, 901-914.

27. Ikeda, F., and Dikic, I. (2008). A typical ubiquitin chains: new molecular signals. ‘Protein Modifications: Beyond the Usual Suspects’ review series. EMBO Rep. 9, 536-542.

28. Kaplan W, et al. Conformational stability of pGEX-expressed Schistosoma japonicum glutathione S-transferase: a detoxification enzyme and fusion-protein affinity tag. Protein Sci 1997; 6:399-406.

29. Kerscher, O., Felberbaum, R., and Hochstrasser, M. (2006). Modification of proteins by ubiquitin and ubiquitin-like proteins. Annu. Rev. Cell Dev. Biol. 22, 159-180.

30. Kim, I., and Rao, H. (2006). What's Ub chain linkage got to do with it? Sci. STKE 2006, pe18.

31. Kim, H., Chen, J., and Yu, X. (2007a). Ubiquitin-binding protein RAP80 mediates BRCA1-dependent DNA damage response. Science 316, 1202-1205.

32. Kim, H. T., Kim, K. P., Lledias, F., Kisselev, A. F., Scaglione, K. M., Skowyra, D., Gygi, S. P., and Goldberg, A. L. (2007b). Certain pairs of ubiquitin-conjugating enzymes (E2s) and ubiquitin-protein ligases (E3s) synthesize nondegradable forked ubiquitin chains containing all possible isopeptide linkages. J. Biol. Chem. 282, 17375-17386.

33. Kirkin V, et al. A role for NBR1 in autophagosomal degradation of ubiquitinated substrates. Mol Cell 2009; 33:505-516.

34. Kolas, N. K., Chapman, J. R., Nakada, S., Ylanko, J., Chahwan, R., Sweeney, F. D., Panier, S., Mendez, M., Wildenhain, J., Thomson, T. M., et al. (2007). Orchestration of the DNA-damage response by the RNF8 ubiquitin ligase. Science 318, 1637-1640.

35. Kozlov G, et al. Structural basis for UBA-mediated dimerization of c-Cbl ubiquitin ligase. J Biol Chem 2007; 282:27547-27555.

36. Lo, Y., Lin, S., Rospigliosi, C. C., Conze, D. B., Wu, C., Ashwell, J. D., Eliezer, D., and Wu, H. (2009). Structural basis for recognition of diubiquitins by NEMO. Mol. Cell, 2009, 33:602-615.

37. Lowe E D, et al. Structures of the Dsk2 UBL and UBA domains and their complex. Acta Crystallogr D Biol Crystallogr 2006; 62:177-188.

38. Maldonado-Baez L, et al. Interaction between Epsin/Yap180 adaptors and the scaffolds Ede1/Pan1 is required for endocytosis. Mol Biol Cell 2008; 19:2936-2948.

39. Mailand, N., Bekker-Jensen, S., Faustrup, H., Melander, F., Bartek, J., Lukas, C., and Lukas, J. (2007). RNF8 ubiquitylates histones at DNA double-strand breaks and promotes assembly of repair proteins. Cell 131, 887-900.

40. Matiuhin Y, et al. Extraproteasomal Rpn10 restricts access of the polyubiquitin-binding protein Dsk2 to proteasome. Mol Cell 2008; 32:415-425.

41. Meiler, J., and Baker, D. (2003). Coupled prediction of protein secondary and tertiary structure. Proc. Natl. Acad. Sci. U.S.A. 21, 12105-12110.

42. Mueller T D, Feigon J. Solution structures of UBA domains reveal a conserved hydrophobic surface for protein-protein interactions. J Mol Biol 2002; 319:1243-1255.

43. Meulmeester, E., Kunze, M., Hsiao, H. H., Urlaub, H., and Melchior, F. (2008). Mechanism and consequences for paralog-specific sumoylation of ubiquitin specific protease 25. Mol. Cell 30, 610-619.

44. Olzmann J A, Chin L S. Parkin-mediated K63-linked polyubiquitination: a signal for targeting misfolded proteins to the aggresome-autophagy pathway. Autophagy 2008; 4:85-87.

45. Peng, J., Schwartz, D., Elias, J. E., Thoreen, C. C., Cheng, D., Marsischky, G., Roelofs, J., Finley, D., and Gygi, S. P. (2003). A proteomics approach to understanding protein ubiquitination. Nat. Biotechnol. 21, 921-926.

46. Peschard P, et al. Structural basis for ubiquitin-mediated dimerization and activation of the ubiquitin protein ligase Cbl-b. Mol Cell 2007; 27:474-485.

47. Pickart, C. M., and Cohen, R. E. (2004). Proteasomes and their kin: proteases in the machine age. Nat. Rev. Mol. Cell Biol. 5, 177-187.

48. Pickart, C. M., and Eddins, M. J. (2004). Ubiquitin: structures, functions, mechanisms. Biochim. Biophys. Acta 1695, 55-72.

49. Pickart, C. M., and Fushman, D. (2004). Polyubiquitin chains: polymeric protein signals. Curr. Opin. Chem. Biol. 8, 610-616.

50. Pickart, C. M., and Raasi, S. (2005). Controlled synthesis of polyubiquitin chains. Methods Enzymol. 399, 21-36.

51. Plans, V., Scheper, J., Soler, M., Loukili, N., Okano, Y., and Thomson, T. M. (2006). The RING finger protein RNF8 recruits UBC13 for lysine 63-based self polyubiquitylation. J. Cell. Biochem. 97, 572-582.

52. Prag G, et al. Mechanism of ubiquitin recognition by the CUE domain of Vps9p. Cell 2003; 113:609-620.

53. Raasi, S., and Pickart, C. M. (2005). Ubiquitin chain synthesis. Methods Mol. Biol. 47-55.

54. Reyes-Turcu, F. E., Shanks, J. R., Komander, D., and Wilkinson, K. D. (2008). Recognition of polyubiquitin isoforms by the multiple ubiquitin binding modules of isopeptidase T. J. Biol. Chem. 283, 19581-19592.

55. Rohl, C. A., Chakrabartty, A., and Baldwin, R. L. (1996). Helix propagation and N-cap propensities of the amino acids measured in alanine-based peptides in 40 volume percent trifluoroethanol. Protein Sci. 12, 2623-2637.

56. Rost, B., Yachdav, G., and Liu, J. (2004). The PredictProtein server. Nucleic Acids Res. Web Server issue, W321-6.

57. Ryabov Y, Fushman D. Interdomain mobility in di-ubiquitin revealed by NMR. Proteins 2006; 63:787-796.

58. Sasaki T, Funakoshi M, Endicott J A, Kobayashi H. Budding yeast Dsk2 protein forms a homodimer via its C-terminal UBA domain. Biochem Biophys Res Commun 2005; 336:530-535.

59. Scheibner K A, Zhang Z. Cole P A. Merging fluorescence resonance energy transfer and expressed protein ligation to analyze protein-protein interactions. Anal Biochem 2003; 317:226-232.

60. Schleif, R., and Wolberger, C. (2004). Arm-domain interactions can provide high binding cooperativity. Protein Sci. 13, 2829-2831.

61. Seibenhener M L, Geetha T, Wooten M W. Sequestosome 1/p62—more than just a scaffold. FEBS Lett 2007; 581:175-179.

62. Seibenhener M L, et al. Sequestosome 1/p62 is a polyubiquitin chain binding protein involved in ubiquitin proteasome degradation. Mol Cell Biol 2004; 24:8055-8068.

63. Sloper-Mould, K. E., Jemc, J. C., Pickart, C. M., and Hicke, L. (2001). Distinct functional surface regions on ubiquitin. J. Biol. Chem. 276, 30483-30489.

64. Sims J J, Cohen R E. Linkage-specific avidity defines the lysine 63-linked polyubiquitin-binding preference of rap80. Mol Cell 2009; 33:775-783.

65. Sobhian, B., Shao, G., Lilli, D. R., Culhane, A. C., Moreau, L. A., Xia, B., Livingston, D. M., and Greenberg, R. A. (2007). RAP80 targets BRCA1 to specific ubiquitin structures at DNA damage sites. Science 316, 1198-1202.

66. Sun, L., and Chen, Z. J. (2004). The novel functions of ubiquitination in signaling. Curr. Opin. Cell Biol. 16, 119-126.

67. Swanson K A, Hicke L, Radhakrishnan I. Structural basis for monoubiquitin recognition by the Ede1 UBA domain. J Mol Biol 2006; 358:713-724.

68. Swanson, K. A., Kang, R. S., Stamenova, S. D., Hicke, L., and Radhakrishnan, I. (2003). Solution structure of Vps27 UIM-ubiquitin complex important for endosomal sorting and receptor downregulation. EMBO J. 22, 4597-4606.

69. Tan J M, et al. Lysine 63-linked ubiquitination promotes the formation and autophagic clearance of protein inclusions associated with neurodegenerative diseases. Hum Mol Genet 2008; 17:431-439.

70. Tan J M, Wong E S, Dawson V L, Dawson T M, Lim K L. Lysine 63-linked polyubiquitin potentially partners with p62 to promote the clearance of protein inclusions by autophagy. Autophagy 2007; 4

71. Trempe, J. F., Brown, N. R., Lowe, E. D., Gordon, C., Campbell, I. D., Noble, M. E., and Endicott, J. A. (2005). Mechanism of Lys48-linked polyubiquitin chain recognition by the Mud1 UBA domain. EMBO J. 24, 3178-3189.

72. Varadan, R., Assfalg, M., Raasi, S., Pickart, C., and Fushman, D. (2005). Structural determinants for selective recognition of a Lys48-linked polyubiquitin chain by a UBA domain. Mol. Cell 18, 687-698.

73. Varadan, R., Assfalg, M., Haririnia, A., Raasi, S., Pickart, C., and Fushman, D. (2004). Solution conformation of Lys63-linked di-ubiquitin chain provides clues to functional diversity of polyubiquitin signaling. J. Biol. Chem. 279, 7055-7063.

74. Varadan R, Walker O, Pickart C, Fushman D. Structural properties of polyubiquitin chains in solution. J Mol Biol 2002; 324:637-647.

75. Wang, B., and Elledge, S. J. (2007). Ubc13/Rnf8 ubiquitin ligases control foci formation of the Rap80/Abraxas/Brca1/Brcc36 complex in response to DNA damage. Proc. Natl. Acad. Sci. USA 104, 20759-20763.

76. Wang, Q., Young, P., and Walters, K. J. (2005). Structure of S5a bound to monoubiquitin provides a model for polyubiquitin recognition. J. Mol. Biol. 348, 727-739.

77. Wang, B., Matsuoka, S., Ballif, B. A., Zhang, D., Smogorzewska, A., Gygi, S. P., and Elledge, S. J. (2007). Abraxas and RAP80 form a BRCA1 protein complex required for the DNA damage response. Science 316, 1194-1198.

78. Wilkinson, K. D. (2004). Quantitative analysis of protein-protein interactions. Methods Mol. Biol. 261, 15-32.

79. Winborn, B. J., Travis, S. M., Todi, S. V., Scaglione, K. M., Xu, P., Williams, A. J., Cohen, R. E., Peng, J., and Paulson, H. L. (2008). The deubiquitinating enzyme ataxin-3, a polyglutamine disease protein, edits lys63 linkages in mixed linkage ubiquitin chains. J. Biol. Chem. 283, 26436-26443.

80. Xu P, et al. Quantitative proteomics reveals the function of unconventional ubiquitin chains in proteasomal degradation. Cell 2009; 137:133-145.

81. Yan, J., and Jetten, A. M. (2008). RAP80 and RNF8, key players in the recruitment of repair proteins to DNA damage sites. Cancer Lett. 271, 179-190.

82. Yan, J., Kim, Y. S., Yang, X. P., Li, L. P., Liao, G., Xia, F., and Jetten, A. M. (2007). The ubiquitin-interacting motif containing protein RAP80 interacts with BRCA1 and functions in DNA damage repair response. Cancer Res. 67, 6647-6656.

83. Zhang D, Raasi S, Fushman D. Affinity makes the difference: nonselective interaction of the UBA domain of Ubiquilin-1 with monomeric ubiquitin and polyubiquitin chains. J Mol Biol 2008; 377:162-180.

We claim:

1. A polypeptide having linkage-specific avidity for K-63 polyubiquitinated proteins comprising at least two ubiquitin binding domains (UBDs) linked to each other by an α-helical amino acid sequence.

2. The polypeptide of claim 1, wherein the amino acid linker comprises about 2 to about 10 amino acids.

3. The polypeptide of claim 2, wherein the amino acid linker comprises 8 amino acids.

4. The polypeptide of claim 2, wherein the amino acid linker comprises 7 amino acids.

5. The polypeptide of claim 1, wherein the UBDs are the same or different.

6. The polypeptide of claim 1, wherein the UBDs are selected from the group consisting of UIM (Ubiquitin Interacting Motif), UBA (Ubiquitin Associated domain), UBM (Ubiquitin Binding Motif), MIU (Motif Interacting with Ubiquitin), DUIM (Double-sided Ubiquitin Interacting Motif), CUE (Coupling of Ubiquitin Conjugation to ER degradation), UBZ (Ubiquitin-Binding Zinc Finger), NZF (Np14 Zinc Finger), A20 ZnF (Zinc Finger), UBP Znf (Ubiquitin-specific Processing Protease Zinc Finger), UEV (Ubiquitin-conjugating Enzyme E2 variant), PFU (PLAA Family Ubiquitin binding), GLUE (GRAM-Like Ubiquitin binding in EAP45), GAT (Golgi-localized, Gamma-ear-containing, Arf-binding), Jab/MPN (Jun kinase Activation domain Binding/Mpr1p and Pad1p N-termini), and a Ubc (Ubiquitin-Conjugating enzyme).

7. The polypeptide of claim 1, wherein the UBDs are UIM.

8. The polypeptide of claim 1, wherein the UBDs are derived from the Rap80 protein or the ataxin-3 protein.

9. The polypeptide of claim 1, wherein said linkage-specific avidity requires no specific contact at or near the isopeptide bond of the polyubiquitinated proteins.

10. A polypeptide having linkage-specific avidity for K-63 polyubiquitinated proteins comprising at least two ubiquitin interacting motifs (UIMs) linked to each other by an amino acid sequence that adopts a helical conformation.

11. The polypeptide of claim 10, wherein the amino acid linker comprises about 2 to about 10 amino acids.

12. The polypeptide of claim 11, wherein the amino acid linker comprises 8 amino acids.

13. The polypeptide of claim 11, wherein the amino acid linker comprises 7 amino acids.

14. The polypeptide of claim 10, wherein the UIMs are the same or different.

15. The polypeptide of claim 10, wherein the UIMs are derived from the Rap80 protein or the ataxin-3 protein.

16. A polypeptide having linkage-specific avidity for K-63 polyubiquitinated proteins comprising at least two ubiquitin interacting motifs (UIMs) linked to each other by an amino acid sequence that reduces flexibility between the UIMs.

17. The polypeptide of claim 16, wherein the amino acid linker comprises about 2 to about 10 amino acids.

18. The polypeptide of claim 17, wherein the amino acid linker comprises 8 amino acids.

19. The polypeptide of claim 17, wherein the amino acid linker comprises 7 amino acids.

20. The polypeptide of claim 16, wherein the UIMs are the same or different.

21. The polypeptide of claim 16, wherein the UIMs are derived from the Rap80 protein or the ataxin-3 protein.

22. The polypeptide of claim 1 further comprising a detection tag.

23. A host cell comprising a polynucleotide sequence encoding the polypeptide of claim 1.

24. A method for isolating K63 polyubiquitinated proteins comprising the steps of contacting the polypeptide of claim 1 with at least one candidate K63 polyubiquitinated protein under conditions allowing the interaction between the UIMs of the polypeptide with the ubiquitin molecules of the candidate K63 polyubiquitinated protein, and detecting the interaction.

25. The method of claim 24, wherein the polypeptide of claim 1 comprises a detectable tag.

26. The method of claim 24, wherein the candidate protein comprises a detectable tag.

27. A polypeptide having linkage-specific avidity for K-48 polyubiquitinated proteins comprising at least two UIMs linked to each other by two amino acids.

28. The polypeptide of claim 27, wherein said UIMs are the same or different.

29. The polypeptide of claim 27, wherein the UIMs are derived from the Rap80 protein or the ataxin-3 protein.

30. A polypeptide having linkage-specific avidity for K-63 polyubiquitinated proteins comprising tandem UIMs linked by a seven amino acid sequence.

31. A polypeptide having linkage-specific avidity for K-48 polyubiquitinated proteins comprising tandem UIMs linked by a two amino acid sequence.


SEQUENCE LISTING (91 sequences)

SEQ ID NO: 1 - protein, 66 aa, Homo sapiens
  misc_feature: Rap80 tandem UIMs (wt)
MPSSHHHHHHSSGLVPRGSHMTEEEQFALALKMSEQEAREVNSQEEEEEELLRKAIAESLNSCRPS

SEQ ID NO: 2 - protein, 58 aa, Homo sapiens
  misc_feature: Rap80 UIM1
MPSSHHHHHHSSGLVPRGSHMTEEEQFALALKMSEQEAREVNSQEEEEEELLNSCRPS

SEQ ID NO: 3 - protein, 58 aa, Homo sapiens
  misc_feature: Rap80 UIM2
MPSSHHHHHHSSGLVPRGSHMTEEEQEQEAREVNSQEEEEEELLRKAIAESLNSCRPS

SEQ ID NO: 4 - protein, 73 aa, Homo sapiens
  misc_feature: Rap80_2xlinker
MPSSHHHHHHSSGLVPRGSHMTEEEQFALALKMSEQEAREVNSQEREVNSQEEEEEELLRKAIAESLNSCRPS

SEQ ID NO: 5 - protein, 102 aa, Homo sapiens
  misc_feature: Vps27 (wt)
MPSSHHHHHHSSGLVPRGSHMDRDYSTPEDEEELIRKAIELSLKESRNSASSEPIVPVVESKNEVKRQEIEEEEDPDLKAAIQESLREAEEAKLRSERQKAC

SEQ ID NO: 6 - protein, 69 aa, Homo sapiens
  misc_feature: Vps27 7aa_linker
MPSSHHHHHHSSGLVPRGSHMEEEEDPDLKAAIQESLREARAEEKVKEDEEELIRKAIELSLKESRNCA

SEQ ID NO: 7 - protein, 65 aa, Homo sapiens
  misc_feature: Rap80_6aa_linker
MPSSHHHHHHSSGLVPRGSHMTEEEQFALALKMSEQEAREVAAEEEEEELLRKAIAESLNSCRPS

SEQ ID NO: 8 - protein, 59 aa, Homo sapiens
  misc_feature: Rap80_nolinker
MPSSHHHHHHSSGLVPRGSHMTEEEQFALALKMSEQEAEEEEELLRKAIAESLNSCRPS

SEQ ID NO: 9 - protein, 66 aa, Homo sapiens
  misc_feature: Rap80_S101E
MPSSHHHHHHSSGLVPRGSHMTEEEQFALALKMSEQEAREVNEQEEEEEELLRKAIAESLNSCRPS

SEQ ID NO: 10 - protein, 66 aa, Homo sapiens
  misc_feature: Rap80_S101A
MPSSHHHHHHSSGLVPRGSHMTEEEQFALALKMSEQEAREVNAQEEEEEELLRKAIAESLNSCRPS

SEQ ID NO: 11 - protein, 66 aa, Homo sapiens
  misc_feature: Rap80_DLKAAIQ
MPSSHHHHHHSSGLVPRGSHMTEEEQFALALKMSEQEADLKAAIQEEEEELLRKAIAESLNSCRPS

SEQ ID NO: 12 - protein, 66 aa, Homo sapiens
  misc_feature: Rap80_ARLEEKV
MPSSHHHHHHSSGLVPRGSHMTEEEQFALALKMSEQEAARLEEKVEEEEELLRKAIAESLNSCRPS

SEQ ID NO: 13 - protein, 63 aa, Homo sapiens
  misc_feature: Ataxin3 (wt)
MPSSHHHHHHSSGLVPRGSHMDEDEEDLQRALALSRQEIDMEDEEADLRRAIQLSMQGSSRNC

SEQ ID NO: 14 - protein, 61 aa, Homo sapiens
  misc_feature: Rap80 ataxin linker
MPSSHHHHHHSSGLVPRGSHMTEEEQFALALKMSEQEADMEEEEELLRKAIAESLNSCRPS

SEQ ID NO: 15 - protein, 7 aa, Homo sapiens
  misc_feature: Rap80 (WT) Linker Sequence
REVNSQE

SEQ ID NO: 16 - protein, 7 aa, Homo sapiens
  misc_feature: Vps27 7aa linker
RAEEKVK

SEQ ID NO: 17 - protein, 7 aa, Homo sapiens
  misc_feature: Rap80 7A linker
AAAAAAA

SEQ ID NO: 18 - protein, 6 aa, Homo sapiens
  misc_feature: Rap80-E Linker Sequence
REVNSQ

SEQ ID NO: 19 - protein, 6 aa, Homo sapiens
  misc_feature: Rap80 6aa linker
REVAAE

SEQ ID NO: 20 - protein, 7 aa, Homo sapiens
  misc_feature: Rap80 S101E Linker Sequence
REVNEQE

SEQ ID NO: 21 - protein, 7 aa, Homo sapiens
  misc_feature: Rap80 S101A Linker Sequence
REVNAQE

SEQ ID NO: 22 - protein, 7 aa, Homo sapiens
  misc_feature: Rap80 DLKAAIQ Linker Sequence
DLKAAIQ

SEQ ID NO: 23 - protein, 7 aa, Homo sapiens
  misc_feature: Rap80 ARLEEKV Linker Sequence
ARLEEKV

SEQ ID NO: 24 - protein, 7 aa, Homo sapiens
  misc_feature: ataxin-3 Rap80 linker
REVNSQE

SEQ ID NO: 25 - protein, 7 aa, Homo sapiens
  misc_feature: ataxin-3 7A linker
AAAAAAA

SEQ ID NO: 26 - protein, 6 aa, Homo sapiens
  misc_feature: Rap80 Linker Sequence
REVAAQ

SEQ ID NO: 27 - protein, 46 aa, Artificial Sequence
  GST Fusion Protein
GSATTPKSLAVEELSGMGFTEEEAHNALEKCNWDLEAATNFLLDSA

SEQ ID NO: 28 - protein, 65 aa, Artificial Sequence
  GST Fusion Protein
GSTGSEYETMLTEIMSMGYERERVVAALRASYNNPHRAVEYLLTGIPGSPEPEHGLGRLERPHRD

SEQ ID NO: 29 - protein, 52 aa, Artificial Sequence
  GST Fusion Protein
GSAGIDHDLIDEFESQGFEKDKIVEVRRLGVKSLDPNDNNTANRIIEELLKS

SEQ ID NO: 30 - protein, 51 aa, Artificial Sequence
  GST Fusion Protein
GSEAGSSKSKAIDHFLAMGFDEEKVVKAIQEHGEDNMEAIANALLSCPEAK

SEQ ID NO: 31 - protein, 66 aa, Artificial Sequence
  His Fusion Protein
MGHHHHHHHHHHSSGHIEGRHMATTPKSLAVEELSGMGFTEEEAHNALEKCNWDLEAATNFLLDSA

SEQ ID NO: 32 - protein, 46 aa, Artificial Sequence
  Fusion Protein
MATTPKSLAVEELSGMGFTEEEAHNALEKCNWDLEAATNFLLDSAG

SEQ ID NO: 33 - protein, 45 aa, Artificial Sequence
  Fusion Protein
MTGSEYETMLTEIMSMGYERERVVAALRASYNNPHRAVEYLLTGG

SEQ ID NO: 34 - protein, 48 aa, Artificial Sequence
  Fusion Protein
MPPEERYEHQLRQLNDMGFFDFDRNVAALRRSGGSVQGALDSLLNGSG

SEQ ID NO: 35 - protein, 43 aa, Artificial Sequence
  Fusion Protein
MTFPEQTIKQLMDLGFPRDAVVKALKQTNGNAEFAASLLFQSG

SEQ ID NO: 36 - protein, 74 aa, Artificial Sequence
  Fusion Protein
MPHHHHHHHHHHSSGHIEGRHMAGIDHDLIDEFESQGFEKDKIVEVLRRLGVKSLDPNDNNTANRIIEELLKSC

SEQ ID NO: 37 - protein, 46 aa, Homo sapiens
  misc_feature: Rap80 tandem UIMs (wt)
MTEEEQFALALKMSEQEAREVNSQEEEEEELLRKAIAESLNSCRPS

SEQ ID NO: 38 - protein, 39 aa, Homo sapiens
  misc_feature: Rap80 single UIM1 (wt)
CMTEEEQFALALKMSEQEAREVNSQEEEEEELLNSSRPS

SEQ ID NO: 39 - protein, 38 aa, Homo sapiens
  misc_feature: Rap80 single UIM2 (wt)
MTEEEQEQEAREVNSQEEEEEELLRKAIAESLNSCRPS

SEQ ID NO: 40 - protein, 50 aa, Homo sapiens
  misc_feature: USP25 tUIM
GDDKDDLQRAIALSLAESNRAFRETGITDEDDKDDLQRAIALSLAESNRA

SEQ ID NO: 41 - protein, 48 aa, Homo sapiens
  misc_feature: EPN3 tUIM
SEEELQLQLALAMSREEAEKPVPPASHRDEDLQLQLALRLSRQEHEKE

SEQ ID NO: 42 - protein, 47 aa, Homo sapiens
  misc_feature: EPS15L1 tUIM
GNEEQQLAWAKRESEKAEQERLARLRRQEQEDLELAIALSKADMPAA

SEQ ID NO: 43 - protein, 47 aa, Homo sapiens
  misc_feature: EPS15 tUIM
PSEEDMIEQAKRESEREEEQRLARLNQQEQEDLELAIALSKSEISEA

SEQ ID NO: 44 - protein, 43 aa, Homo sapiens
  misc_feature: ANKRD13D tUIM
PSFEEQLRLALELSSREQEERERRGQQEEEDLQRILQLSLTEH

SEQ ID NO: 45 - protein, 46 aa, Homo sapiens
  misc_feature: EPN2 tUIM
GEEELQLQLALAMSREVAEQEERLRRGDDLRLQMALEESRRDTVKI

SEQ ID NO: 46 - protein, 46 aa, Homo sapiens
  misc_feature: EPN1,13 tUIM
GEEELQLQLALAMSKEEADQEERIRRGDDLRLQMAIEESKRETGGK

SEQ ID NO: 47 - protein, 46 aa, Homo sapiens
  misc_feature: EPN1,12 tUIM
GEEELQLQLALAMSKEEADQPPSCGPEDDAQLQLALSLSREEHDKE

SEQ ID NO: 48 - protein, 46 aa, Homo sapiens
  misc_feature: EPN1,23 tUIM
PEDDAQLQLALSLSREEHDKEERIRRGDDLRLQMAIEESKRETGGK

SEQ ID NO: 49 - protein, 45 aa, Homo sapiens
  misc_feature: ZFAND2B tUIM
LSEDEALQRALEMSLAETKPQVPSCQEEEDLALAQALSASEAEYQ

SEQ ID NO: 50 - protein, 44 aa, Homo sapiens
  misc_feature: PARP10 tUIM
LEEEAALQLALHRSLEPQGQVAEQEEAAALRQALTLSLLEQPPL

SEQ ID NO: 51 - protein, 43 aa, Homo sapiens
  misc_feature: USP37 tUIM
EREEQELQQALAQSLQEQEAWEQKEDDDLKRATELSLQEFNNS

SEQ ID NO: 52 - protein, 41 aa, Homo sapiens
  misc_feature: ATXN3 tUIM
DEDEEDLQRALALSRQEIDMEDEEADLRRAIQLSMQGSSRN

SEQ ID NO: 53 - protein, 41 aa, Homo sapiens; identical to SEQ ID NO: 52
  misc_feature: Human Ataxin-3 tUIM

SEQ ID NO: 54 - protein, 41 aa, Macaca mulatta; identical to SEQ ID NO: 52
  misc_feature: Macaque Ataxin-3 tUIM

SEQ ID NO: 55 - protein, 41 aa, Pongo pygmaeus; identical to SEQ ID NO: 52
  misc_feature: Orangutan Ataxin-3 tUIM

SEQ ID NO: 56 - protein, 41 aa, Pan troglodytes; identical to SEQ ID NO: 52
  misc_feature: Chimpanzee Ataxin-3 tUIM

SEQ ID NO: 57 - protein, 41 aa, Canis familiaris
  misc_feature: Dog Ataxin-3 tUIM
DEDEEDLQRALALSRQEIDMEDEEADLRRAIQLSMQGSSRS

SEQ ID NO: 58 - protein, 41 aa, Bos primigenius
  misc_feature: Cow Ataxin-3 tUIM
DEDEEELQRALALSRQEIDMEDEEADLRRAIQLSMQGTSRN

SEQ ID NO: 59 - protein, 41 aa, Mus musculus
  misc_feature: Mouse Ataxin-3 tUIM
DEDEDDLQRALAISRQEIDMEDEEADLRRAIQLSMQGSSRS

SEQ ID NO: 60 - protein, 41 aa, Rattus norvegicus
  misc_feature: Rat Ataxin-3 tUIM
DDDEDDLQRALAMSRQEIDMEDEEADLRRAIQLSMQGSSRG

SEQ ID NO: 61 - protein, 41 aa, Gallus gallus
  misc_feature: Chicken Ataxin-3 tUIM
DEDEENFQRALALSRQEIDMEDEEADLRRAIQLSMQGSRQS

SEQ ID NO: 62 - protein, 41 aa, Oryzias latipes
  misc_feature: Medaka Ataxin-3 tUIM
VDDDEELKRALALSRQEMDVEDEEADLRRAIQLSMQESDSS

SEQ ID NO: 63 - protein, 41 aa, Lagocephalus gloveri
  misc_feature: Fugu Ataxin-3 tUIM
GEEDEELKRALALSRQDIDVEDEEADLRRAIQLSMQGEDLN

SEQ ID NO: 64 - protein, 41 aa, Gasterosteus aculeatus
  misc_feature: Stickleback Ataxin-3 tUIM
VDEDDELKKALALSRQDIDVEDEEADLRRAIQLSMQAGSAA

SEQ ID NO: 65 - protein, 41 aa, Xenopus tropicalis
  misc_feature: X. tropicalis Ataxin-3 tUIM
DEDDDNLQKALALSRQQIDMEDEEADLRRAIQLSMQGGHQS

SEQ ID NO: 66 - protein, 41 aa, Danio rerio
  misc_feature: Zebrafish Ataxin-3 tUIM
DEDEEELRKALALSRQDLQVEDEEADLRRAIQMSMQGSSDA

SEQ ID NO: 67 - protein, 46 aa, Ochotona princeps
  misc_feature: Pika Rap80 tUIM
MTEEEQFALALKMSEQEAREANNQEEEEEQLLRKAIAESLNXXXXX

SEQ ID NO: 68 - protein, 46 aa, Sciurus griseus
  misc_feature: Squirrel Rap80 tUIM
MTEEEQFALALKMSEQEAREVNSQEEEEEELLRKAIAESLNXXXXX

SEQ ID NO: 69 - protein, 45 aa, Erinaceus concolor
  misc_feature: Hedgehog Rap80 tUIM
MTEEEQFALALKMSEQEAREVNSQEEEEEALLRKAIAESLNSWRS

SEQ ID NO: 70 - protein, 46 aa, Canis familiaris
  misc_feature: Dog Rap80 tUIM
MTEEEQFALALKMSEQEAREMNSQEEEEEELLRKAIAESLNSCRPS

SEQ ID NO: 71 - protein, 46 aa, Felis catus; identical to SEQ ID NO: 70
  misc_feature: Cat Rap80 tUIM

SEQ ID NO: 72 - protein, 46 aa, Cavia porcellus
  misc_feature: Guinea pig Rap80 tUIM
MTEEEQFALALKMSEQEAKEMNNQEEEEEELLRKAIAESLNSCRPS

SEQ ID NO: 73 - protein, 46 aa, Mus musculus
  misc_feature: Mouse Rap80 tUIM
MSEEEQFALALKMSEQEAREVNNQEEKEEELLRKAIAESLNSCWSS

SEQ ID NO: 74 - protein, 46 aa, Oryctolagus cuniculus
  misc_feature: Rabbit Rap80 tUIM
MTEEEQFALALKMSEQEAREVNNQEEEEEELLRKAIAESLNSCRPS

SEQ ID NO: 75 - protein, 42 aa, Elephas maximus
  misc_feature: Elephant Rap80 tUIM
MTEEEQFALALKMSEQEAREVNSQEEEEEKAIAESLNTCRPS

SEQ ID NO: 76 - protein, 46 aa, Rousettus aegyptiacus; identical to SEQ ID NO: 37
  misc_feature: Microbat Rap80 tUIM

SEQ ID NO: 77 - protein, 46 aa, Sus scrofa; identical to SEQ ID NO: 37
  misc_feature: Pig Rap80 tUIM

SEQ ID NO: 78 - protein, 46 aa, Pongo pygmaeus; identical to SEQ ID NO: 37
  misc_feature: Orangutan Rap80 tUIM

SEQ ID NO: 79 - protein, 46 aa, Pan troglodytes; identical to SEQ ID NO: 37
  misc_feature: Chimpanzee Rap80 tUIM

SEQ ID NO: 80 - protein, 46 aa, Microcebus murinus; identical to SEQ ID NO: 37
  misc_feature: Mouse Lemur Rap80 tUIM

SEQ ID NO: 81 - protein, 46 aa, Macaca mulatta; identical to SEQ ID NO: 37
  misc_feature: Macaque Rap80 tUIM

SEQ ID NO: 82 - protein, 46 aa, Homo sapiens; identical to SEQ ID NO: 37
  misc_feature: Human Rap80 tUIM

SEQ ID NO: 83 - protein, 46 aa, Equus caballus; identical to SEQ ID NO: 37
  misc_feature: Horse Rap80 tUIM

SEQ ID NO: 84 - protein, 46 aa, Larus fuscus; identical to SEQ ID NO: 37
  misc_feature: Lesser Rap80 tUIM

SEQ ID NO: 85 - protein, 46 aa, Bos primigenius
  misc_feature: Cow Rap80 tUIM
MTEEEQFALALKMSEQEAREVNSQEAEEEELLRKAIAESLNSCRPS

SEQ ID NO: 86 - protein, 46 aa, Gallus gallus
  misc_feature: Chicken Rap80 tUIM
MTEEEQFALALKMSEQEARQVNCQEEEEEELLRKAIAESLNSCQPL

SEQ ID NO: 87 - protein, 46 aa, Didelphis marsupialis
  misc_feature: Opossum Rap80 tUIM
MTEEEQFALALKMSEQEAQQVNDQAEEEEALLRKAIAESLNSCQPS

SEQ ID NO: 88 - protein, 46 aa, Otolemur garnetti
  misc_feature: Bushbaby Rap80 tUIM
MTEEEQFALALKMSEQEAREVNSQEEEEEELLRKAIAESLNSCQPS

SEQ ID NO: 89 - protein, 46 aa, Suncus murinus
  misc_feature: Shrew Rap80 tUIM
MTEEEQFELALQMSEQEARAVNSQEEEEEALLRKAIAESLNSCRPS

SEQ ID NO: 90 - protein, 44 aa, Tupaia belangeri
  misc_feature: Treeshrew Rap80 tUIM
MTEEEQFALALRMSEQEAREVNSQEEEEEELLRKAIAESLSSCS

SEQ ID NO: 91 - protein, 47 aa, Tetraodon fluviatilis
  misc_feature: Tetraodon Rap80 tUIM
MTEEEMIDLALRLSEQEASIVAIRRQKEEEEAMMKAIQDSVSTKWLT